(19) United States
(12) Patent Application Publication
(10) Pub. No.: US 2022/0370467 A1
(43) Pub. Date: Nov. 24, 2022

(54) USING OF A PPAR-DELTA AGONIST IN THE TREATMENT OF KIDNEY DISEASE

(71) Applicants: Reneo Pharmaceuticals, Inc., Irvine, CA (US); WASHINGTON UNIVERSITY, St. Louis, MO (US)

(72) Inventors: Colin O'CARROLL, San Diego, CA (US); Niall O'DONNELL, San Diego, CA (US); Jeffrey MINER, St. Louis, MO (US)

(21) Appl. No.: 17/761,515

(22) PCT Filed: Sep. 18, 2020
(86) PCT No.: PCT/US2020/051458

§ 371 Date: Mar. 17, 2022

Related U.S. Application Data
(60) Provisional application No. 62/903,539, filed on Sep. 20, 2019.

(51) Int. Cl. A61K 31/5375; A61P 13/12; A61K 31/192; A61K 45/06

(52) U.S. Cl. CPC A61K 31/5375; A61P 13/12; A61K 31/192; A61K 45/06

(57) ABSTRACT

Described herein is the use of a PPAR-delta agonist in the treatment of kidney diseases, wherein: the kidney disease is Alport syndrome, Goodpasture syndrome, thin basement membrane nephropathy (TBMN), focal segmental glomerulosclerosis (FSGS), benign familial hematuria (BFH), post-transplant anti-GBM (Glomerular Basement Membrane) nephritis, X-linked Alport syndrome (XLAS), autosomal recessive Alport syndrome (ARAS) or autosomal dominant Alport syndrome (ADAS).

CROSS-REFERENCE

This application claims the benefit of U.S. Provisional Patent Application No. 62/903,539 filed on Sep. 20, 2019, which is incorporated herein by reference in its entirety.

### FIELD OF THE INVENTION

Described herein are methods of using a peroxisome proliferator-activated receptor delta (PPARδ) agonist in the treatment or prevention of kidney diseases or disorders.

### BACKGROUND OF THE INVENTION

Mitochondrial biogenesis and its attendant processes enhance metabolic pathways such as fatty acid oxidation (FAO) and increase antioxidant defense mechanisms that ameliorate injury from aging, tissue hypoxia, and glucose or fatty acid overload, all of which contribute to the pathogenesis of acute and chronic kidney disease. PPARδ, a member of the nuclear regulatory superfamily of ligand-activating transcriptional regulators, is expressed throughout the body, including the kidneys. PPARδ agonists induce genes related to fatty acid oxidation and mitochondrial biogenesis. PPARδ also has anti-inflammatory properties.

### SUMMARY OF THE INVENTION

In one aspect, described herein is a method for treating kidney disease in a mammal, comprising administering to the mammal a peroxisome proliferator-activated receptor delta (PPARδ) agonist, wherein the mammal has one of more mutations in the genes encoding α3, α4, or α5 chains of collagen IV.

In some embodiments, the PPARδ agonist binds to and activates the cellular PPARδ and does not substantially activate the cellular peroxisome proliferator activated receptors -alpha (PPARα) and -gamma (PPARγ).

In some embodiments, the kidney disease is Alport syndrome, Goodpasture syndrome, thin basement membrane nephropathy (TBMN), focal segmental glomerulosclerosis (FSGS), benign familial hematuria (BFH), post-transplant anti-GBM (Glomerular Basement Membrane) nephritis

In some embodiments, the kidney disease is X-linked Alport syndrome (XLAS), autosomal recessive Alport syndrome (ARAS) or autosomal dominant Alport syndrome (ADAS).

In some embodiments, the PPARδ agonist increases fatty acid oxidation (FAO) in kidney tissues, increases carnitine palmitoyl-transferase 1(CPT1) levels in kidney tissues, attenuates excessive collagen deposition in kidney tissues, increase mitochondrial function in kidney tissues, attenuate oxidative stress in kidney tissues, decrease inflammation in kidney tissues, or a combination thereof.

In some embodiments, the PPARδ agonist compound is a phenoxyalkylcarboxylic acid compound; or a pharmaceutically acceptable salt thereof. In some embodiments, the PPARδ agonist compound is a phenoxyethanoic acid compound, phenoxypropanoic acid compound, phenoxybutanoic acid compound, phenoxypentanoic acid compound, phenoxyhexanoic acid compound, phenoxyoctanoic acid compound, phenoxynonanoic acid compound, or phenoxydecanoic acid compound; or a pharmaceutically acceptable salt thereof. In some embodiments, the PPARδ agonist compound is a phenoxyethanoic acid compound or a phenoxyhexanoic acid compound; or a pharmaceutically acceptable salt thereof. In some embodiments, the PPARδ agonist compound is an allyloxyphenoxyethanoic acid acid compound; or a pharmaceutically acceptable salt thereof

In some embodiments, the PPARδ agonist is (E) [4,-[3-(4-Fluorophenyl)-3-[4-[3-(morpholin-4-yl)propynyl]phenyl]allyloxy]-2-methyl-phenoxy]acetic acid (Compound 1) having the following structure:

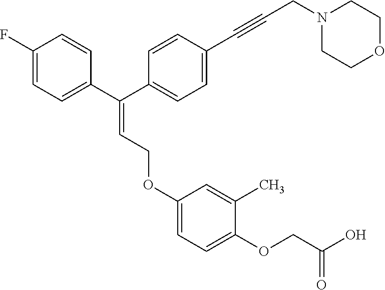 [SMILES: Cc1cc(OC/C=C(/c2ccc(F)cc2)c2ccc(C#CCN3CCOCC3)cc2)ccc1OCC(=O)O]

or a pharmaceutically acceptable salt thereof

In some embodiments, the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 10 mg to about 500mg, about 50 mg to about 200 mg, or about 75 mg to about 125 mg.

In another aspect, described herein is a method for increasing fatty acid oxidation (FAO), increasing carnitine palmitoyl-transferase 1(CPT1) levels, attenuating excessive collagen deposition, increasing mitochondrial function, increasing mitochondrial biogenesis, attenuating oxidative stress, decreasing inflammation, or a combination thereof, in the kidneys of a mammal with kidney disease, comprising administering a peroxisome proliferator-activated receptor delta (PPARδ) agonist to the mammal. In some embodiments, the PPARδ agonist binds to and activates the cellular PPARδ and does not substantially activate the cellular peroxisome proliferator activated receptors -alpha (PPARα) and -gamma (PPARγ). In some embodiments, the mammal has one of more mutations in the genes encoding α3, α4, or a 5 chains of collagen IV. In some embodiments, the kidney disease is Alport syndrome, Goodpasture syndrome, thin basement membrane nephropathy (TBMN), focal segmental glomerulosclerosis (FSGS), benign familial hematuria (BFH), post-transplant anti-GBM (Glomerular Basement Membrane) nephritis. In some embodiments, the kidney disease is X-linked Alport syndrome (XLAS), autosomal recessive Alport syndrome (ARAS) or autosomal dominant Alport syndrome (ADAS). In some embodiments, the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof.

In another aspect, described herein is a method for treating kidney disease in a mammal, comprising administering to the mammal (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, wherein the kidney disease is polycystic kidney disease (PKD), IgA nephropathy (Bergers Disease), diabetic nephropathy, focal segmental glomerulosclerosis (FSGS), Fabry Disease, Alport syndrome, Glomerulonephritis, Goodpasture syndrome, thin basement membrane nephropathy (TBMN), Nephrotic Syndrome, focal segmental glomerulosclerosis (FSGS), benign familial hematuria (BFH), post-transplant anti-GBM (Glomerular Basement Membrane) nephritis, chronic kidney disease (CKD) or acute kidney injury.

In another aspect, described herein is a method for treating kidney fibrosis in a mammal, comprising administering to the mammal (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof.

In some embodiments, (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, is administered to the mammal at a dose of about 10 mg to about 500 mg, about 50 mg to about 200 mg, or about 75 mg to about 125 mg.

In some embodiments, the method comprises lowering of urine protein levels, reducing proteinuria, reducing intraglomerular pressure, ameliorating glomerular injury, ameliorating extracellular matrix deposition, reducing renal fibrosis, arresting a decline in the estimated glomerular filtration rate (eGFR), increasing eGFR, delaying the onset of end-stage renal disease (ESRD), or combinations thereof.

In some embodiments, the method comprises achieving a urine protein:creatinine ratio of less than about 0.5 mg/mg if the baseline value is greater than about 1.0 mg/mg.

In some embodiments, the method comprises achieving an about 50% reduction of urine protein:creatinine ratio if the baseline value is greater than about 0.2 but less than about 1.0.

In some embodiments, a PPARδ agonist (e.g. Compound 1, or a pharmaceutically acceptable salt thereof), is systemically administered to the mammal with a kidney disease. In some embodiments, the PPARδ agonist is administered to the mammal orally, by injection or intraveneously. In some embodiments, the PPARδ agonist is administered to the mammal in the form of an oral solution, oral suspension, powder, pill, tablet or capsule.

In one aspect, described herein is a pharmaceutical composition comprising PPARδ agonist and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In one aspect, described herein is a method of treating or preventing any one of the kidney diseases or conditions described herein comprising administering a therapeutically effective amount of a PPARδ agonist to a mammal in need thereof.

In any of the aforementioned aspects are further embodiments in which the effective amount of the PPARδ agonist (e.g. Compound 1, or a pharmaceutically acceptable salt thereof), is: (a) systemically administered to the mammal; and/or (b) administered orally to the mammal; and/or (c) intravenously administered to the mammal; and/or (d) administered by injection to the mammal; and/or (e) adminstered non-systemically or locally to the mammal.

In any of the aforementioned aspects are further embodiments comprising single administrations of the effective amount of the PPARδ agonist (e.g. Compound 1, or a pharmaceutically acceptable salt thereof), including further embodiments in which the PPARδ agonist (e.g. Compound 1, or a pharmaceutically acceptable salt thereof), is administered once a day to the mammal or is administered to the mammal multiple times over the span of one day. In some embodiments, the PPARδ agonist (e.g. Compound 1, or a pharmaceutically acceptable salt thereof), is administered on a continuous dosing schedule. In some embodiments, the PPARδ agonist is administered on a continuous daily dosing schedule.

In any of the aforementioned aspects involving the treatment of a disease or condition are further embodiments comprising administering at least one additional agent in addition to the administration of a PPARδ agonist (e.g. Compound 1, or a pharmaceutically acceptable salt thereof). In various embodiments, each agent is administered in any order, including simultaneously.

In some embodiments, the at least one additional therapeutic agent is a Nicotinamide Adenine Dinucleotide (NAD+) pathway modulator.

In some embodiments, the at least one additional therapeutic agent is a Poly ADP Ribose Polymerase (PARP) modulator, Aminocarboxymuconate Semialdehyde Decarboxylase (ACMSD) modulator or N′-Nicotinamide Methyltransferase (NNMT) modulator.

In some embodiments, the at least one additional therapeutic agent is an inhibitor of the renin-angiotensin-aldosterone system (RAAS).

In some embodiments, the at least one additional therapeutic agent is an angiotensin-converting enzyme (ACE) inhibitor, angiotensin-receptor blocker (ARB), aldosterone inhibitor, calcineurin inhibitor, TGF-β1 inhibitor, matrix metalloproteinase inhibitor, vasopeptidase A inhibitor or HMG-CoA reductase inhibitor, chemokine receptor 1 blocker. In some embodiments, the angiotensin converting enzyme (ACE) inhibitor is Benazepril, Cilazapril, Enalapril, Fosinopril, Lisinopril, Perinopril, Ramapril, Quinapril, or Trandolapril. In some embodiments, the ARB is Candesartan, Epresartan, Irbesartan, Losartan, Telmisartan, or Valsartan. In some embodiments, the aldosterone inhibitor is Spironolactone.

In any of the embodiments disclosed herein, the mammal is a human.

In some embodiments, the PPARδ agonist (e.g. Compound 1, or a pharmaceutically acceptable salt thereof), is administered to a human. In some embodiments, the PPARδ agonist (e.g. Compound 1, or a pharmaceutically acceptable salt thereof), is orally administered.

Articles of manufacture, which include packaging material, a compound described herein, or a pharmaceutically acceptable salt thereof, within the packaging material, and a label that indicates that a PPARδ agonist (e.g. Compound 1, or a pharmaceutically acceptable salt thereof), is used for modulating the activity of PPARδ, or for the treatment, prevention or amelioration of one or more symptoms of a kidney disease or condition that would benefit from modulation of PPARδactivity, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 shows that administration of Compound 1 to B 6129S1 hybrid Col4a3^{-/-} mice significantly reduced reduced proteinuria at 17-weeks of age, which is the late stage of kidney disease.

FIG. 2 shows that administration of Compound 1 to B 6129S1 hybrid Col4a3^{-/-} mice suppressed the increase of blood urea nitrogen (BUN) at 12 and 17-weeks of age.

FIG. 3A shows improvements in renal histology on B 6129S hybrid Col4a3^{-/-} mice with Compound 1. Necrotic regions in the cortex was decreased in Compound 1-treated B 6129S1 hybrid Col4a3^{-/-} mice compared with vehicle-treated mice.

FIG. 3B shows improvements in renal histology on B 6129S hybrid Col4a3^{-/-} mice with Compound 1. The extent of fibrosis decreased in Compound 1 treated B 6129S1 hybrid Col4a3^{-/-} mice compared with vehicle-treated mice.

FIG. 4 shows the effect of Compound 1 treatment in Study 1 on the expression of inflammatory and fibrosis-related molecules in whole kidneys.

### DETAILED DESCRIPTION

Healthy mitochondria are vital to normal cellular activities. Mitochondrial dysfunction drives the pathogenesis of a wide variety of medical disorders, including acute conditions and chronic diseases. Distinct aspects of mitochondrial function, for example, bioenergetics, dynamics, and cellular signaling are well described and impairments in these activities likely contribute to disease pathogenesis. In some embodiments, impairments of mitochondrial function, for example, bioenergetics, dynamics, and cellular signaling, contribute to kidney disease pathogenesis.

Mitochondria play a myriad of roles in cellular homeostasis and defects in mitochondrial function can lead to a broad spectrum of diseases. These functional defects are apparent in tissues that have a high energy demand such as the kidneys. In some embodiments, diseases associated with mitochondrial dysfunction manifest as kidney disease.

Treatments that improve mitochondrial function in kidneys are useful in the treatment of kidney disease.

In some embodiments, mitochondrial-directed therapies, such as treatment with a PPARδ agonist, have the ability to address multiple molecular abnormalities simultaneously and prove to be more efficacious than compounds that target an isolated protein.

One of the mechanisms cells use to manage protein requirements is gene regulation.

Mitochondrial health depends on several complex processes that help cells function under normal and stress conditions. Mitochondrial dysfunction can lead to mitochondrial clearance (mitophagy) or cell death (apoptosis). Mitochondria are dynamic organelles, able to change their morphology by undergoing fusion or fission. Many diseases, such as but not limited to kidney disease, stress mitochondria which can disrupt the normal dynamics processes.

Scarring of the internal organs caused by microscopic injury is known as fibrosis. It is characterized by uncontrolled deposition of matrix and basement membrane structural proteins in inappropriate places, often in the virtual spaces between functioning units of the organ. Organ fibrosis, a final common pathway of chronic or iterative tissue injury, is an inappropriate wound-healing response and is frequently associated with inflammation (inflammatory cells), loss of organ function, and tissue ischemia resulting from abnormal angiogenesis. Fibrosis contributes both directly and indirectly to organ demise and that the cells that lay down matrix, known as myofibroblasts, perpetuate the fibrotic process. Organ fibrosis is seen in many common and rare diseases including diabetes mellitus, ischemic heart disease, hypertension, and chronic diseases of lung, liver, kidney, gut, heart, and brain. The kidney is particularly susceptible to fibrosis, perhaps because of its highly unusual vascular bed and predisposition to ischemia.

“Fibrosis,”as used herein, refers to the accumulation of extracellular matrix constituents that occurs following trauma, inflammation, tissue repair, immunological reactions, cellular hyperplasia, and neoplasia.

In some embodiments, disclosed herein is a method of reducing fibrosis in a tissue (e.g. kidney tissue) comprising contacting a fibrotic cell or tissue with a compound disclosed herein, in an amount sufficient to decrease or inhibit the fibrosis. In some embodiments, the fibrosis includes a fibrotic condition.

In some embodiments, reducing fibrosis, or treatment of a fibrotic condition, includes reducing or inhibiting one or more of: formation or deposition of extracellular matrix proteins; the number of pro-fibrotic cell types (e.g., fibroblast or immune cell numbers); cellular collagen or hydroxyproline content within a fibrotic lesion; expression or activity of a fibrogenic protein; or reducing fibrosis associated with an inflammatory response.

Kidney fibrosis is characterized by loss of capillary networks, accumulation of fibrillary collagens, activated myofibroblasts and inflammatory cells. In fibrosis, tubular epithelial cells are lost due to cell death and the remaining cells dedifferentiate leading to reduced expression of characteristic epithelial markers and increased expression of mesenchymal markers. While tubular epithelial cells may not be the direct precursors of myofibroblasts, they play an instrumental role in orchestrating fibrosis by multiple mechanisms including secreting different cytokines.

Alterations in cellular metabolism, including changes in fuel source preferences (glucose, fatty acids or ketones) is an important mechanism of cell differentiation. Tubular epithelial cells have high levels of baseline energy consumption and a copious supply of mitochondria. Fatty acid oxidation (FAO) is the preferred energy source for highly metabolic cells because it generates more ATP than does oxidation of glucose. Metabolism of fatty acids requires their transport into the mitochondria, which is mediated by carnitine palmitoyl-transferase 1(CPT1) and this enzyme conjugates fatty acids with carnitine. CPT1 is considered to be the rate-limiting enzyme in FAO.

Alterations in cellular metabolism have been observed in fibrotic kidneys, and enzymes and regulators of FAO are reduced in kidneys from human subjects with chronic kidney disease and in mouse models of kidney fibrosis. PPARδ is a key transcription factors that regulates the expression of proteins involved in fatty acid uptake and oxidation. Healthy renal tubular epithelial cells primarily rely on FAO as their energy source. In some embodiments, lower FAO by tubular epithelial cells contributes to tubulointerstitial fibrosis development. In some embodiments, a PPARδ agonist restores FAO in fibrotic kidneys to pre-fibrotic levels. In some embodiments, a PPARδ agonist increases FAO in fibrotic kidneys. In some embodiments, a PPARδ agonist increases CPT1 levels and increases FAO.

In some embodiments, a PPARδ agonist is used in the treatment of kidney fibrosis. Kidney fibrosis can result from various diseases and insults to the kidneys. Examples of such diseases and insults include chronic kidney disease, metabolic syndrome, vesicoureteral reflux, tubulointerstitial renal fibrosis, IgA nephropathy, diabetes (including diabetic nephropathy), Alport syndrome, and resultant glomerular nephritis (GN), including, but not limited to, focal segmental glomerulosclerosis and membranous glomerulonephritis, mesangiocapillary GN.

Glomerulonephritis, which causes inflammation in glomeruli, is a common cause of end-stage renal failure. Severe and prolonged inflammation can damage glomeruli and lead to kidney fibrosis. Connective tissue growth factor (CTGF) is a member of the CCN matricellular protein family, consisting of four domains, that regulates the signaling of other growth factors and promotes kidney fibrosis. In some embodiments, a PPARδ agonist contemplated in any of the methods disclosed herein for the treatment of kidney disease does not induce CTGF. In some embodiments, excessive collagen deposition in kidney tissues is attenuated with a PPARδ agonist.

It has become recognized that metabolic syndrome is a cluster of abnormalities including diabetic hallmarks such as insulin resistance, as well as central or visceral obesity and hypertension. In nearly all cases, dysregulation of glucose results in the stimulation of cytokine release and upregulation of extracellular matrix deposition. Additional factors contributing to chronic kidney disease, diabetes, metabolic syndrome, and glomerular nephritis include hyperlipidemia, hypertension, and proteinuria, all of which result in further damage to the kidneys and further stimulate the extracellular matrix deposition. Thus, regardless of the primary cause, insults to the kidneys may result in kidney fibrosis and the concomitant loss of kidney function. (Schena, F. and Gesualdo, L., Pathogenic Mechanisms of Diabetic Nephropathy, J. Am. Soc. Nephrol., 16: S30-33(2005); Whaley-Connell, A., and Sower, J. R., Chronic Kidney Disease and the Cardiometabolic Syndrome, J. Clin. Hypert., 8(8): 546-48(2006)).

In some embodiments, a PPARδ agonist is used in the treatment of kidney disease. In some embodiments, the kidney disease is kidney fibrosis. In some embodiments, the kidney disease is Alport renal disease. In some embodiments, the kidney disease is chronic kidney disease.

### Alport Syndrome

Alport syndrome is a genetic condition characterized by kidney disease, hearing loss, and eye abnormalities. Individuals with Alport syndrome experience progressive loss of kidney function. Almost all affected individuals have blood in their urine (hematuria), which indicates abnormal functioning of the kidneys, and many individuals with Alport syndrome also develop high levels of protein in their urine (proteinuria). The kidneys become less able to function as this condition progresses, resulting in end-stage renal disease (ESRD).

People with Alport syndrome frequently develop sensorineural hearing loss, which is caused by abnormalities of the inner ear, during late childhood or early adolescence. Affected individuals may also have misshapen lenses in the eyes (anterior lenticonus) and abnormal coloration of the light-sensitive tissue at the back of the eye (retina). These eye abnormalities seldom lead to vision loss.

Significant hearing loss, eye abnormalities, and progressive kidney disease are more common in males with Alport syndrome than in affected females. In some embodiments, a PPARδ agonist is used in the treatment of Alport syndrome in a male human.

Mutations in the COL4A3, COL4A4, and COL4A5 genes cause Alport syndrome. These genes each provide instructions for making one component of a protein called type IV collagen. This protein plays an important role in the kidneys, specifically in structures called glomeruli. Glomeruli are clusters of specialized blood vessels that remove water and waste products from blood and create urine. Mutations in these genes result in abnormalities of the type IV collagen in glomeruli, which prevents the kidneys from properly filtering the blood and allows blood and protein to pass into the urine. As a result, the integrity of the glomerular filtration barrier is disrupted, resulting in initial glomerular hemodynamic changes and, thereafter, progressive glomerular and tubulointerstitial fibrosis accompanied by severe inflammation. Gradual scarring of the kidneys occurs, eventually leading to kidney failure in many people with Alport syndrome.

Type IV collagen is also an important component of inner ear structures, particularly the organ of Corti, that transform sound waves into nerve impulses for the brain. Alterations in type IV collagen often result in abnormal inner ear function, which can lead to hearing loss. In the eye, this protein is important for maintaining the shape of the lens and the normal color of the retina. Mutations that disrupt type IV collagen can result in misshapen lenses and an abnormally colored retina.

Alport syndrome can have different inheritance patterns. About 80 percent of cases are caused by mutations in the COL4A5 gene and are inherited in an X-linked pattern. This gene is located on the X chromosome, which is one of the two sex chromosomes. In males (who have only one X chromosome), one altered copy of the COL4A5 gene in each cell is sufficient to cause kidney failure and other severe symptoms of the disorder. In females (who have two X chromosomes), a mutation in one copy of the COL4A5 gene usually only results in hematuria, but some women experience more severe symptoms. A characteristic of X-linked inheritance is that fathers cannot pass X-linked traits to their sons.

In approximately 15 percent of cases, Alport syndrome results from mutations in both copies of the COL4A3 or COL4A4 gene and is inherited in an autosomal recessive pattern. The parents of an individual with the autosomal recessive form of this condition each have one copy of the mutated gene and are called carriers. Some carriers are unaffected and others develop a less severe condition called thin basement membrane nephropathy, which is characterized by hematuria.

Alport syndrome has autosomal dominant inheritance in about 5 percent of cases. People with this form of Alport syndrome have one mutation in either the COL4A3 or COL4A4 gene in each cell. It remains unclear why some individuals with one mutation in the COL4A3 or COL4A4 gene have autosomal dominant Alport syndrome and others have thin basement membrane nephropathy.

Alport syndrome is also known as congenital hereditary hematuria, hematuria-nephropathy-deafness syndrome, hematuric hereditary nephritis, hemorrhagic familial nephritis, hemorrhagic hereditary nephritis, hereditary familial congenital hemorrhagic nephritis, hereditary hematuria syndrome, hereditary interstitial pyelonephritis and hereditary nephritis.

The 3 genetic types of Alport syndrome are: XLAS (X-linked Alport syndrome), ARAS (autosomal recessive Alport syndrome) and ADAS (autosomal dominant Alport syndrome). XLAS results from mutations of the alpha-5 chain type IV collagen (gene COL4A5) . ARAS is caused by mutations in the alpha-3 or alpha-4 chains (genes COL4A3 or COL4A4) . ADAS is caused by mutations in the alpha-3 or alpha-4 chains (genes COL4A3 or COL4A4) .

### X-Linked Alport Syndrome (XLAS)

Males have one X and one Y chromosome and females have two X chromosomes. X-linked Alport syndrome is caused by mutations in the COL4A5 gene, which resides on the X chromosome. X-linked disorders cause more severe symptoms in affected males than in affected females because males have only one X chromosome.

Males with XLAS are severely affected and always develop kidney failure sometime in their lives, because they do not have a normal copy of the gene to buffer the effect of the mutant gene. Females, who have two X chromosomes, have two copies of the COL4A5 gene. In girls with XLAS, one copy of the gene carries a mutation, but the other copy is normal. The normal copy of the gene counters the effect of the mutation, so that girls with XLAS usually have milder symptoms than boys. However, girls with X-linked Alport syndrome can also develop kidney failure and should not be considered as only carriers of XLAS.

A male with XLAS will pass the affected X chromosome gene to all of his daughters and they will have XLAS. A male cannot pass an X-linked gene to his sons because the Y chromosome (not the X chromosome) is always passed to male offspring. A female with XLAS has a 50% chance with each pregnancy of having an affected child.

### Autosomal Recessive Alport Syndrome (ARAS)

Autosomal recessive disorders result when both copies of a gene are defective. Typically, each parent of a child with a recessive condition passes a mutant gene to the affected child. The genes COL4A3 and COL4A4 are located on chromosome 2. Each person has two copies of this chromosome, and two copies of both the COL4A3 and COL4A4 genes. The parents only have one mutation in one of the chromosomes and so they can have no symptoms or have some hematuria (blood in the urine). However, they will not have progression of the disease.

Unlike X-linked Alport syndrome, the autosomal recessive type affects females just as severely as males.

### Autosomal Dominant Alport Syndrome (ADAS)

About 5% of people with Alport syndrome have ADAS. These people have one mutant copy of the COL4A3 or COL4A4 gene. Mutation in one copy of COL4A3 or COL4A4 can cause progressive kidney disease and hearing loss. People with ADAS resemble people with XLAS, with some differences: kidney failure occurs relatively late in life (after age 40), changes in the eyes are very unusual and there is no difference in severity of disease in males and females. People with ADAS usually have a family history that is positive for progressive kidney disease and hearing loss. Mutation in one copy of COL4A3 or COL4A4 can also cause thin basement membrane nephropathy (TBMN), which differs from ADAS in that progressive kidney disease and hearing loss are very unusual. People with TBMN usually have a family history that is negative for progressive kidney disease and hearing loss. Researchers are still trying to understand why some people with these mutations have ADAS and others have TBMN.

### COL4A3 Gene

The COL4A3 gene provides instructions for making one component of type IV collagen, which is a flexible protein. The COL4A3 gene makes the alpha 3(IV) chain of type IV collagen. This chain combines with two other types of alpha (IV) chains (the alpha4 and alpha5 chains) to make a complete type IV collagen molecule. Type IV collagen molecules attach to each other to form complex protein networks. These networks make up a large portion of basement membranes, which are thin sheet-like structures that separate and support cells in many tissues. Type IV collagen alpha3-4-5 networks play an especially important role in the basement membranes of the kidney, inner ear, and eye.

More than 40 mutations in the COL4A3 gene have been found to cause Alport syndrome. Most of these mutations change single protein building blocks (amino acids) in a region where the alpha3(IV) collagen chain combines with other type IV collagen chains. Other mutations in the COL4A3 gene severely decrease or prevent the production of alpha 3(IV) chains. As a result, there is a serious deficiency of the type IV collagen alpha3-4-5 network in the basement membranes of the kidney, inner ear, and eye. In the kidney, other types of collagen accumulate in the basement membranes, eventually leading to scarring of the kidneys and kidney failure. Mutations in this gene can also lead to abnormal function in the inner ear, resulting in hearing loss.

Mutations in the COL4A3 gene have been found to cause thin basement membrane nephropathy. This condition typically causes people to have blood in their urine (hematuria) but no other signs or symptoms of kidney disease. In the past, this condition was often called benign familial hematuria. Thin basement membrane nephropathy rarely progresses to kidney failure.

Goodpasture syndrome is a severe disease of the lungs and the kidneys caused by antibodies to the alpha 3(IV) collagen chains. Antibodies are immune system proteins that normally attack foreign substances such as bacteria or viruses, but in Goodpasture syndrome, they target alpha 3(IV) collagen chains. It remains unclear why some people make antibodies to their own collagen chains. The antibodies cause inflammation when they attach (bind) to the basement membranes of blood vessels in the air sacs (alveoli) of the lungs and filtering units (glomeruli) of the kidneys. As a result, people with Goodpasture syndrome can develop kidney failure and bleeding in the lungs, which causes them to cough up blood. In some people, antibodies attack only the kidneys. These people are said to have anti-glomerular basement membrane nephritis.

### COL4A4 Gene

The COL4A4 gene provides instructions for making one component of type IV collagen, which is a flexible protein. Specifically, this gene makes the alpha4(IV) chain of type IV collagen. This chain combines with two other types of alpha (IV) chains (the alpha3 and alpha5 chains) to make a complete type IV collagen molecule. Type IV collagen molecules attach to each other to form complex protein networks. These networks make up a large portion of basement membranes, which are thin sheet-like structures that separate and support cells in many tissues. Type IV collagen alpha3-4-5 networks play an especially important role in the basement membranes of the kidney, inner ear, and eye.

More than 20 mutations in the COL4A4 gene have been found to cause Alport syndrome. Most of these mutations change single protein building blocks (amino acids) in a region where the alpha4(IV) collagen chain combines with other type IV collagen chains. Other mutations in the COL4A4 gene severely decrease or prevent the production of alpha4(IV) chains. As a result, there is a serious deficiency of the type IV collagen alpha3-4-5 network in the basement membranes of the kidney, inner ear, and eye. In the kidney, other types of collagen accumulate in the basement membranes, eventually leading to scarring of the kidneys and kidney failure. Mutations in this gene can also lead to abnormal function in the inner ear, resulting in hearing loss.

Mutations in the COL4A4 gene have been found to cause thin basement membrane nephropathy. This condition typically causes people to have blood in their urine (hematuria) but no other signs or symptoms of kidney disease. In the past, this condition was often called benign familial hematuria. Thin basement membrane nephropathy rarely progresses to kidney failure.

### COL4A5 Gene

The COL4A5 gene provides instructions for making one component of type IV collagen, which is a flexible protein. Specifically, this gene makes the alpha5(IV) chain of type IV collagen. This chain combines with two other types of alpha (IV) chains (the alpha3 and alpha4 chains) to make a complete type IV collagen molecule. Type IV collagen molecules attach to each other to form complex protein networks. These networks make up a large portion of basement membranes, which are thin sheet-like structures that separate and support cells in many tissues. Type IV collagen alpha3-4-5 networks play an especially important role in the basement membranes of the kidney, inner ear, and eye.

More than 400 mutations in the COL4A5 gene have been found to cause Alport syndrome. Most of these mutations change single protein building blocks (amino acids) in a region where the alpha5(IV) collagen chain combines with other type IV collagen chains. Other mutations in the COL4A5 gene severely decrease or prevent the production of alpha5(IV) chains. As a result, there is a serious deficiency of the type IV collagen alpha3-4-5 network in the basement membranes of the kidney, inner ear, and eye. In the kidney, other types of collagen accumulate in the basement membranes, eventually leading to scarring of the kidneys and kidney failure. Mutations in this gene can also lead to abnormal function in the inner ear, resulting in hearing loss.

### Ear Fibrosis

In some embodiments, a PPARδ agonist is used in the treatment of ear fibrosis or a disease or condition associated with ear fibrosis. Like kidney fibrosis, ear fibrosis can result from various diseases and insults to the ears. Fibrosis can occur in the middle ear as well as the inner ear. Inflammation in the middle ear can result in medial canal fibrosis and this is characterized by the formation of fibrotic tissue in the bony external auditory meatus (Ishii, Fluid and Fibrosis in the Human Middle Ear, Am. J. Otolaryngol, 1985: 6: 196-199). Fibrosis of the inner ear include disorders where strial dysfunction resulting from membrane thickening is observed. These diseases include Alport syndrome, lupus and diabetes. Type IV collagen disorders (as seen with Alport syndrome patients) is associated with sensorineural hearing loss with structural changes in the connective tissue and micromechanics of the inner ear. Detailed assessments of basement membrane morphology have been measured in the mouse model of Alport syndrome which shows clear thickening of the basemement membrande of the stria vascularis (Cosgrove, Ultrastructural, physiological, and molecular defects in the inner ear of a gene-knockout mouse model for autosomal Alport syndrome. Hear Res 1998; 121:84-98).

### Ischemic Acute Kidney Injury (AKI)

Ischemic acute kidney injury (AKI) is characterized by persistent proximal tubule mitochondrial dysfunction. Due to their highly oxidative metabolism, proximal tubule cells utilize fatty acids to generate the energy required for their specialized function.

In some embodiments, provided herein is a method of enhancing fatty acid oxidation in a mammal with a PPARδ agonist. In some embodiments, enhancing fatty acid oxidation in restores mitochondrial function, offering a potential therapeutic treatment for AKI.

Compound 1 was evaluated in the Goldblatt's 2 kidney 1 clip (2K1C) rat animal model of renovascular hypertension which is characterized by ischemic nephropathy of the clipped kidney (Fedorova et al., 2013). Clipped kidneys from untreated rats developed tubular and glomerular necrosis and massive interstitial, periglomerular and perivascular fibrosis. Compound 1 treated kidneys did not exhibit any histochemical features of necrosis; fibrotic lesions were present only in perivascular areas. Necrosis in the untreated clipped kidneys was associated with an increased oxidative stress, up regulation and mitochondrial translocation of the pro-death protein BNIP3 specifically in tubules. In the kidneys of Compound 1-treated rats oxidative stress was attenuated and BNIP3 protein decreased notably in the mitochondrial fraction when compared to untreated animals. In untreated clipped kidneys, mitochondria were dysfunctional as revealed by perturbations in the levels of MCAD, COXIV, TFAM, and Parkin proteins and AMPK activation, while in Compound 1-treated rats these proteins remained at the physiological levels. Nuclear amounts of oxidative stress-responsive proteins, NRF1 and NRF2 were below physiological levels in treated kidneys. Mitochondrial biogenesis and autophagy were inhibited similarly in both treated and untreated 2K 1C kidneys as indicated by a decrease in PGC1-a and deficiency of the autophagy-essential proteins LC3-II and ATG5.

Exaggerated oxidative stress is a disturbance in the regular function of cells. In order to control the oxidative stress level, cells must balance pro- and antioxidant systems. Regarding the kidney physiology, the main principle of proper redox regulation is to maintain the balance of electrolytes and physiological buffer systems to keep renal functions. Additionally, kidneys remove a whole range of toxins and waste metabolites, which otherwise would accumulate in the organism inducing an imbalance in redox homeostasis. Furthermore, oxidative stress contributes to and worsens a wide variety of kidney diseases.

In some embodiments, a PPARδ agonist is used to attenuate oxidative stress in the kidneys of a mammal with kidney disease.

Abnormal metabolism, decreased ATP levels, increased ROS production, and chronic inflammatory signaling are common features of kidney diseases. When left unresolved, these pathologic processes can lead to abnormal cellular proliferation, tissue fibrosis and remodeling, and kidney damage. In some embodiments, mitochondrial dysfunction, oxidative stress, and inflammation are features of kidney diseases. In some embodiments, a PPARδ agonist is used to increase mitochondrial function, attenuate oxidative stress, and decrease inflammation in the kidneys of a mammal with kidney disease.

Described herein is the use of a PPARδ agonist in the treatment of kidney disease in a mammal. In some embodiments, the kidney disease is chronic kidney disease (CKD). In some embodiments, the mammal has a mutation in the a 3 chain of collagen IV.

Both acute and chronic kidney disease, regardless of initiating cause (infection, diabetes, hypertension, autoimmunity), have inflammation and immune activation in common. In some embodiments, a PPARδ agonist targets these common inflammatory pathways that are implicated in kidney disease.

In some embodiments, a PPARδ agonist activates molecular pathways that promote the resolution of inflammation by restoring mitochondrial function, increasing fatty acid oxidation, reducing oxidative stress, and inhibiting pro-inflammatory signaling.

In the kidney, the first stage of the blood filtering process takes place in the glomerulus, which consists of a small tuft of capillaries containing endothelial cells, between which are large pores, and mesangial cells which are modified smooth muscle cells that lie between the capillaries. Tight coordination between these cell types is necessary for proper filtration. The pores between the endothelial cells allow for the free filtration of fluid, plasma solutes, and protein. When endothelial cells become dysfunctional, due to oxidative stress or other reasons, the pores can become more permeable and increase spillage of protein, which can drive further inflammatory signaling and oxidative stress. The mesangial cells regulate blood flow by their contractile activity, and contraction of the cells reduces surface area for filtration of the blood. Mesangial cells also remove proteins and other molecules trapped in the glomerular basement membrane, or filtration barrier.

In some embodiments, a PPARδ agonist described herein reverses endothelial dysfunction and chronic, disease-related mesangial cell contraction, resulting in increased surface area of the glomerulus and increased GFR. In some embodiments, a PPARδ agonist inhibits activation of inflammatory and pro-fibrotic pathways that lead to structural remodeling and glomerulosclerosis.

As described above, Alport syndrome is caused by mutations in the genes encoding type IV collagen (α3, α4, α5), a major structural component of the glomerular basement membrane (GBM) in the kidney. Progressive loss of the filtration barrier allows excessive proteinuria, which ultimately leads to end-stage kidney disease (ESKD). Patients with Alport syndrome are normally diagnosed with the disease in childhood to early adulthood and have average glomerular filtration rate (GFR) declines of 4.0 mL/min/1.73 m² per year. The progressive decline of GFR in Alport syndrome patients leads to renal failure and end-stage renal disease (ESRD). Fifty percent of males with the most prevalent subtype of Alport syndrome require dialysis or kidney transplant by age 25. The incidence of renal failure in these patients increases to 90% by age 40 and nearly 100% by age 60. Similar to patients with other forms of CKD, Alport syndrome patients receiving dialysis are at increased risk for cardiovascular disease and infections, which are the most common causes of death in these patients. Currently, there are no approved therapies for the treatment of Alport syndrome. In some embodiments, a PPARδ agonist described herein is used to increase kidney function in Alport syndrome patients as measured by estimated GFR (eGFR).

In another embodiment, described herein is a method of reducing the rate of decrease in mitochondrial biogenesis in one or more kidney tissues of a subject relative to a control, wherein the rate of decrease in mitochondrial biogenesis comprises a comparison of one or more measurements of mitochondrial biogenesis in the subject to a baseline measurement of mitochondrial biogenesis in the same subject. In another embodiment, reducing the rate of decrease in mitochondrial biogenesis in the subject comprises a return to the subjects baseline measurement of mitochondrial biogenesis faster than the control. In a further embodiment, reducing the rate of decrease in mitochondrial biogenesis in the subject comprises a return to the subjects baseline measurement of mitochondrial biogenesis following a period of disuse in less than 95%, or less than 90%, or less than 85%, or less than 80%, or less than 75%, or less than 70%, or less than 65%, or less than 60%, or less than 55%, or less than 50% of the time to return to baseline for a control. In another embodiment, the decrease in mitochondrial biogenesis in the subject is less than the decrease in mitochondrial biogenesis relative to the control. In a further embodiment, the decrease in mitochondrial biogenesis in the subject comprises less than a 50%, less than a 45%, less than a 40%, less than a 35%, less than a 30%, less than a 25%, less than a 20%, less than a 15%, less than a 10%, less than a 9%, less than an 8%, less than a 7%, less than a 6%, less than a 5%, less than a 4%, less than a 3%, less than a 2%, less than a 1%, or a 0% decrease in mitochondrial biogenesis relative to the subjects baseline measurement of mitochondrial biogenesis prior to a period of disuse.

Mitochondrial biogenesis is measured by mitochondrial mass and volume through histological section staining using a fluorescently labeled antibody specific to the oxidative-phosphorylation complexes, such as the Anti-OxPhox Complex Vd subunit antibody from Life Technologies or using mitochondrial specific dyes in live cell staining, such as the Mito-tracker probes from Life Technologies. Mitochondrial biogenesis can also be measured by monitoring the gene expression of one or more mitochondrial biogenesis related transcription factors such as PGC1a, NRF1 , or NRF2 using a technique such as QPCR.

In some aspects of the invention, PPARδ agonist is administered in a therapeutically effective amount to a subject (e.g., a human). As used herein, the term “effective amount” or “therapeutically effective amount” refers to an amount of an active ingredient that elicits the desired biological or medicinal response, for example, reduction or alleviation of the symptoms of the condition being treated. In some embodiments of the invention, the amount of PPARδ agonist administered can vary depending on various factors, including, but not limited to, the weight of the subject, the nature and/or extent of the subjects condition, etc.

### Compounds

A peroxisome proliferator activated receptor—delta (PPARδ) agonist is a fatty acid, lipid, protein, peptide, small molecule, or other chemical entity that binds to the cellular PPARy and elicits a downstream response, namely gene transcription, either native gene transcription or a reporter construct gene transcription, comparable to endogenous ligands such as retinoic acid or comparable to a standard reference PPARδ agonist such as carbacyclin.

In an embodiment, a PPARδ agonist is a selective agonist. As used herein, a selective PPARδ agonist is viewed as a chemical entity that binds to and activates the cellular PPARδ and does not substantially activate the cellular peroxisome proliferator activated receptors alpha (PPARγ) and gamma (PPARδ). As used herein, a selective PPARδ agonist is a chemical entity that has at least a 10-fold maximum activation (as compared to endogenous receptor ligand) with a greater than 100-fold potency for activation of PPARδ relative to either or both of PPARα and PPARγ. In a further embodiment, a selective PPARδ agonist is a chemical entity that binds to and activates the cellular human PPARδ and does not substantially activate either or both of human PPARα and PPARyγIn a further embodiment, a selective PPARδ agonist is a chemical entity that has at least a 10 fold, or a 20 fold, or a 30 fold, or a 40 fold, or a 50 fold, or a 100 fold potency for activation of PPARδ relative to either or both of PPARδ and PPARγ.

“Activation” here is defined as the abovementioned downstream response, which in the case of PPAR's is gene transcription. Gene transcription may be measured indirectly as downstream production of proteins reflective of the activation of the particular PPAR subtype under study. Alternatively, an artificial reporter construct may be employed to study the activation of the individual PPAR's expressed in cells. The ligand binding domain of the particular receptor to be studied may be fused to the DNA binding domain of a transcription factor, which produces convenient laboratory readouts, such as the yeast GAL4 transcription factor DNA binding domain. The fusion protein may be transfected into a laboratory cell line along with a Gal4 enhancer, which effects the expression of the luciferase protein. When such a system is transfected into a laboratory cell line, binding of a receptor agonist to the fusion protein will result in light emission.

A selective PPARδ agonist may exemplify the above gene transcription profile in cells selectively expressing PPARy, and not in cells selectively expressing PPARγ or PPARα. In an embodiment, the cells may be expressing human PPARδ, PPARγ, and PPARα, respectively.

In a further embodiment, a PPARδ agonist may have an EC50 value of less than 500 μm as determined by the PPAR transient transactivation assay described below. In an embodiment, the EC50 value is less than 1 μμm. In another embodiment, the EC50 value is less than 500 nM. In another embodiment, the EC50 value is less than 100 nM. In another embodiment, the EC50 value is less than 50 nM.

The PPAR transient transactivation assay may be based on transient transfection into human HEK293 cells of two plasmids encoding a chimeric test protein and a reporter protein respectively. The chimeric test protein may be a fusion of the DNA binding domain (DBD) from the yeast GAL4 transcription factor to the ligand binding domain (LBD) of the human PPAR proteins. The PPAR-LBD moiety harbored in addition to the ligand binding pocket also has the native activation domain, allowing the fusion protein to function as a PPAR ligand dependent transcription factor. The GAL4 DBD will direct the chimeric protein to bind only to Gal4 enhancers (of which none existed in HEK293 cells). The reporter plasmid contained a Gal4 enhancer driving the expression of the firefly luciferase protein. After transfection, HEK293 cells expressed the GAL4-DBD-PPAR-LBD fusion protein. The fusion protein will in turn bind to the Gal4 enhancer controlling the luciferase expression, and do nothing in the absence of ligand. Upon addition to the cells of a PPAR ligand, luciferase protein will be produced in amounts corresponding to the activation of the PPAR protein. The amount of luciferase protein is measured by light emission after addition of the appropriate substrate.

Cell Culture and Transfection: HEK293 cells may be grown in DMEM+10% FCS. Cells may be seeded in 96-well plates the day before transfection to give a confluency of 50-80% at transfection. A total of 0.8 mg DNA containing 0.64 mg pM1a/gLBD, 0.1 mg pCMVbGa1, 0.08 mg pGL2(Ga14)₅, and 0.02 mg pADVANTAGE may be transfected per well using FuGene transfection reagent according to the manufacturers instructions. Cells may be allowed to express protein for 48 h followed by addition of compound.

Plasmids: Human PPARδ may be obtained by PCR amplification using cDNA synthesized by reverse transcription of mRNA from human liver, adipose tissue, and plancenta, respectively. Amplified cDNAs may be cloned into pCR2.1 and sequenced. The ligand binding domain (LBD) of each PPAR isoform may be generated by PCR (PPARδ: aa 128—C-terminus) and fused to the DNA binding domain (DBD) of the yeast transcription factor GAL4 by subcloning fragments in frame into the vector pM1 (Sadowski et al. (1992), Gene 118, 137), generating the plasmids pM1αLBD, pM1δyLBD, and pM1δ. Ensuing fusions may be verified by sequencing. The reporter may be constructed by inserting an oligonucleotide encoding five repeats of the GAL4 recognition sequence (Webster et al. (1988), Nucleic Acids Res. 16, 8192) into the vector pGL2 promotor (Promega), generating the plasmid pGL2(GAL4)₅. pCMVbGa1 may be purchased from Clontech and pADVANTAGE may be purchased from Promega.

Compounds: Compounds may be dissolved in DMSO and diluted 1:1000 upon addition to the cells. Compounds may be tested in quadruple in concentrations ranging from 0.001 to 300 μM. Cells may be treated with compound for 24 h followed by luciferase assay. Each compound may be tested in at least two separate experiments.

Luciferase assay: Medium including test compound may be aspirated and 100 μl PBS including 1 mM Mg⁺⁺ and Ca⁺⁺ may be added to each well. The luciferase assay may be performed using the LucLite kit according to the manufacturers instructions (Packard Instruments). Light emission may be quantified by counting on a Packard LumiCounter. To measure β-galactosidase activity, 25 ml supernatant from each transfection lysate may be transferred to a new microplate. β-Galactosidase assays may be performed in the microwell plates using a kit from Promega and read in a Labsystems Ascent Multiscan reader. The β-galactosidase data may be used to normalize (transfection efficiency, cell growth, etc.) the luciferase data.

Statistical methods: The activity of a compound may be calculated as fold induction compared to an untreated sample. For each compound, the efficacy (maximal activity) may be given as a relative activity compared to Wy14,643 for PPARα, rosiglitazone for PPARγ, and carbacyclin for PPARδ. The EC50 is the concentration giving 50% of maximal observed activity. EC50 values may be calculated via non-linear regression using GraphPad PRISM 3.02 (GraphPad Software, San Diego, Calif.).

In a further embodiment, a PPARδ agonist has a molecular weight of less than 1000 g/mol, or a molecular weight of less than 950 g/mol, or a molecular weight of less than 900 g/mol, or a molecular weight of less than 850 g/mol, or a molecular weight of less than 800 g/mol, or a molecular weight of less than 750 g/mol, or a molecular weight of less than 700 g/mol, or a molecular weight of less than 650 g/mol, or a molecular weight of less than 600 g/mol, or a molecular weight of less than 550 g/mol, or a molecular weight of less than 500 g/mol, or a molecular weight of less than 450 g/mol, or a molecular weight of less than 400 g/mol, or a molecular weight of less than 350 g/mol, or a molecular weight of less than 300 g/mol, or a molecular weight of less than 250 g/mol. In another embodiment, a PPARδ agonist has a molecular weight of greater than 200 g/mol, or a molecular weight of greater than 250 g/mol, or a molecular weight of greater than 250 g/mol, or a molecular weight of greater than 300 g/mol, or a molecular weight of greater than 350 g/mol, or a molecular weight of greater than 400 g/mol, or a molecular weight of greater than 450 g/mol, or a molecular weight of greater than 500 g/mol, or a molecular weight of greater than 550 g/mol, or a molecular weight of greater than 600 g/mol, or a molecular weight of greater than 650 g/mol, or a molecular weight of greater than 700 g/mol, or a molecular weight of greater than 750 g/mol, or a molecular weight of greater than 800 g/mol, or a molecular weight of greater than 850 g/mol, or a molecular weight of greater than 900 g/mol, or a molecular weight of greater than 950 g/mol, or a molecular weight of greater than 1000 g/mol. Any of the upper and lower limits described above in this paragraph may be combined.

In some embodiments, a PPARδ agonist is a PPARδ agonist compound disclosed in any of the following published patent applications: WO 97/027847, WO 97/027857, WO 97/028115, WO 97/028137, WO 97/028149, WO 98/027974, WO 99/004815, WO 2001/000603, WO 2001/025181, WO 2001/025226, WO 2001/034200, WO 2001/060807, WO 2001/079197, WO 2002/014291, WO 2002/028434, WO 2002/046154, WO 2002/050048, WO 2002/059098, WO 2002/062774, WO 2002/070011, WO 2002/076957, WO 2003/016291, WO 2003/024395, WO 2003/033493, WO 2003/035603, WO 2003/072100, WO 2003/074050, WO 2003/074051, WO 2003/074052, WO 2003/074495, WO 2003/084916, WO 2003/097607, WO 2004/000315, WO 2004/000762, WO 2004/005253, WO 2004/037776, WO 2004/060871, WO 2004/063165, WO 2004/063166, WO 2004/073606, WO 2004/080943, WO 2004/080947, WO 2004/092117, WO 2004/092130, WO 2004/093879, WO 2005/060958, WO 2005/097098, WO 2005/097762, WO 2005/097763, WO 2005/115383, WO 2006/055187, WO 2007/003581, and WO 2007/071766 (each of which is incorporated for such PPARδ agonist compounds).

In some embodiments, a PPARδ agonist is a PPARδ agonist compound disclosed in any of the following published patent applications: WO 2014/165827; WO 2016/057660; WO 2016/057658; WO 2017/180818; WO 2017/062468; and WO 2018/067860 (each of which is incorporated for such PPARδ agonist compounds).

In some embodiments, a PPARδ agonist is a PPARδ agonist compound disclosed in any of the following published patent applications: United States Patent Application Publication Nos. 20160023991, 201 70226154, 20170304255, and 20170305894 (each of which is incorporated for such PPARδ agonist compounds).

In some embodiments, a PPARδ agonist compound is a phenoxyalkylcarboxylic acid compound. In some embodiments, the phenoxyalkylcarboxylic acid compound is a 2-methylphenoxyalkylcarboxylic acid compound.

In some embodiments, a PPARδ agonist compound is a phenoxyalkylcarboxylic acid compound that is a phenoxyethanoic acid compound, phenoxypropanoic acid compound, phenoxypropenoic acid compound, phenoxybutanoic acid compound, phenoxybutenoic acid compound, phenoxypentanoic acid compound, phenoxypentenoic acid compound, phenoxyhexanoic acid compound, phenoxyhexenoic acid compound, phenoxyoctanoic acid compound, phenoxyoctenoic acid compound, phenoxynonanoic acid compound, phenoxynonenoic acid compound, phenoxydecanoic acid compound, or phenoxydecenoic acid compound. In some embodiments, a PPARδ agonist compound is a phenoxyethanoic acid compound or a phenoxyhexanoic acid compound. In some embodiments, a PPARδ agonist compound is a phenoxyethanoic acid compound. In some embodiments, the phenoxyethanoic acid compound is a 2-methylphenoxyethanoic acid compound. In some embodiments, a PPARδ agonist compound is a phenoxyhexanoic acid compound.

In some embodiments, a PPARδ agonist compound is a phenoxyethanoic acid compound, a ((benzamidomethyl)phenoxy)hexanoic acid compound, a ((heteroarylmethyl)phenoxy)hexanoic acid compound, a methylthiophenoxyethanoic acid compound, or an allyloxyphenoxyethanoic acid acid compound.

In some embodiments, a PPARδ agonist compound is a ((benzamidomethyl)phenoxy)hexanoic acid compound.

In some embodiments, a PPARδ agonist compound is a ((heteroarylmethyl)phenoxy)hexanoic acid compound. In some embodiments, a PPARδ agonist compound is a ((imidazolylmethyl)phenoxy)hexanoic acid compound. In some embodiments, a PPARδ agonist compound is an imidazol-1-ylmethylphenoxyhexanoic acid compound. In some embodiments, a PPARδ agonist compound is a 6-(2-((2-phenyl-1H-imidazol-1-yl)methyl)phenoxy)hexanoic acid.

In some embodiments, a PPARδ agonist compound is an allyloxyphenoxyethanoic acid compound. In some embodiments, the allyloxyphenoxyethanoic acid compound is a 4-allyloxy-2-methylphenoxy)ethanoic acid compound.

In some embodiments, a PPARδ agonist compound is a methylthiophenoxyethanoic acid compound. In some embodiments, a PPARδ agonist compound is a 4-(methylthio)phenoxy)ethanoic acid compound.

In some embodiments, a PPARδ agonist compound is a phenoxyalkylcarboxylic acid compound selected from the group consisting of: (Z)-[2-Methyl-4-[3-(4-methylphenyl)-[3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid (Compound 1); (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; and {4-[3,3-Bis-(4-bromo-phenyl)-allyloxy]-2-methyl-phenoxy}-acetic acid; (R)-3-methyl-6-(2-((5-methyl-2-(4-(trifluoromethyl)phenyl)-1H-imidazol-1-yl)methyl)phenoxy)hexanoic acid; (R)-3-methyl-6-(2-((5-methyl-2-(6-(trifluoromethyl)pyridin-3-yl)-1H-imidazol-1-yl)methyl)phenoxy)hexanoic acid; (E)-[4-[3-(4-Fluorophenyl)-3-[4-[3-(morpholin-4-yl)propynyl]phenyl]allyloxy]-2-methyl-phenoxy]acetic acid (Compound 1); 2-{4-[({2-[2-[Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954); 2-[2-methyl-4-[[3-methyl-4-[[4-(trifluoromethyl)phenyl]methoxy]phenyl]thio]phenoxy]-acetic acid; 2-[2-methyl-4-[[[4-methyl-2-[4-(trifluoromethyl)phenyl]-5-thiazolyl]methyl]thio]phenoxy]-acetic acid (GW-501516); [4-[[[2-[3-Fluoro-4-(trifluoromethyl)phenyl]-4-methyl-5-thiazolyl]methyl]thio]-2-methylphenoxy]acetic acid (GW0742 also known as GW610742); 2-[2,6 dimethyl-4-[3-[4-(methylthio)phenyl]-3-oxo-1(E)-propenyl]phenoxyl]-2-methylpropanoic acid (elafibranor; GFT-505); {2-methyl-4-[5-methyl-2-(4-trifluoromethyl-phenyl)-2H-[1,2,3]triazol-4-ylmethylsulfanyl]-phenoxy}-acetic acid; and [4-({(2R)-2-Ethoxy-3-[4-(trifluoromethyl)phenoxy]propyl}sulfanyl)-2-methylphenoxy]acetic acid (seladelpar; MBX-8025); (S)-4-[cis-2,6-dimethyl-4-(4-trifluoromethoxy-phenyl)piperazine-1-sulfonyl]-indan-2-carboxylic acid or a tosylate salt thereof (KD-3010); (2s)-2-{4-butoxy-3-[(({[2-Fluoro-4-(Trifluoromethyl)phenyl]carbonyl}amino)methyl]benzyl}butanoic acid (TIPP-204); [4-[3-(4-Acetyl-3-hydroxy-2-propylphenoxy)propoxy]phenoxy]acetic acid (L-165,0411); 2-(4-{2-[(4-Chlorobenzoyl)amino]ethyl}phenoxy)-2-methylpropanoic acid (bezafibrate); or a pharmaceutically acceptable salt thereof.

In another embodiment, a PPARδ agonist is a 2-methylphenoxyalkylcarboxylic acid compound selected from the group consisting of (E)-[4-[3-(4-Fluorophenyl)-3-[4-[3-(morpholin-4-yl)propynyl]phenyl]allyloxy]-2-methyl-phenoxy]acetic acid (Compound 1); 2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954); 2-[2-methyl-4-[[3-methyl-4-[[4-(trifluoromethyl)phenyl]methoxy]phenyl]thio]phenoxy]-acetic acid; 2-[2-methyl-4-[[[4-methyl-2-[4-(trifluoromethyl)phenyl]-5-thiazolyl]methyl]thio]phenoxy]-acetic acid (GW-501516); [4-[[[2-[3-Fluoro-4-(trifluoromethyl)phenyl]-4-methyl-5-thiazolyl]methyl]thio]-2-methylphenoxy]acetic acid (GW0742 also known as GW610742); 2-[2,6 dimethyl-4-4-[3-[4-(methylthio)phenyl]-3-oxo-1(E)-propenyl]phenoxyl]-2-methylpropanoic acid (elafibranor; GFT-505); {2-methyl-4-[5-methyl-2-(4-trifluoromethyl-phenyl)-2H-[1,2,3]triazol-4-ylmethylsulfanyl]-phenoxy}-acetic acid; and [4-({(2R)-2-Ethoxy-3-[4-(trifluoromethyl)phenoxy]propyl}sulfanyl)-2-methylphenoxy]acetic acid (seladelpar; MBX-8025).

In another embodiment, a PPARδ agonist is a compound selected from the group consisting of (S)-4-[cis-2,6-dimethyl-4-(4-trifluoromethoxy-phenyl)piperazine-1-sulfonyl]-indan-2-carboxylic acid or a tosylate salt thereof (KD-3010); (2s)-2-{4-butoxy-3-[({[2-Fluoro-4-(Trifluoromethyl)phenyl]carbonyl}amino)methyl]benzyl}butanoic acid (TIPP-204); [4-[3-(4-Acetyl-3-hydroxy-2-propylphenoxy)propoxy]phenoxy]acetic acid (L-165,0411); and 2-(4-{2-[(4-Chlorobenzoyl)amino]ethyl}phenoxy)-2-methylpropanoic acid (bezafibrate).

In another embodiment, a PPARδ agonist is a compound selected from the group consisting of sodelglitazar; lobeglitazone; netoglitazone; and isaglitazone; 2-[2-methyl-4-[[3-methyl-4-[[4-(trifluoromethyl)phenyl]methoxy]phenyl]thio]phenoxy]-acetic acid (See WO 2003/024395); (S)-4-[cis-2,6-dimethyl-4-(4-trifluoromethoxy-phenyl)piperazine-1-sulfonyl]-indan-2-carboxylic acid or a tosylate salt thereof (KD-3010); 4-butoxy-a-ethyl-3-[[[2-fluoro-4-(trifluoromethyl)benzoyl]amino]methyl]-benzenepropanoic acid (TIPP-204); 2-[2-methyl-4-[[[4-methyl-2-[4-(trifluoromethyl)phenyl]-5-thiazolyl]methyl]thio]phenoxy]-acetic acid (GW-501516); 2-[2,6 dimethyl-4-[3-[4-(methylthio)phenyl]-3-oxo-1(E)-propenyl]phenoxyl]-2-methylpropanoic acid (GFT-505); and {2-methyl-4-[5-methyl-2-(4-trifluoromethyl-phenyl)-2H-[1,2,3]triazol-4-ylmethylsylfanyl]-phenoxy}-acetic acid.

In some embodiments, a PPARδ agonist is (E)-[4-[3-(4-Fluorophenyl)-3-[4-[3-(morpholin-4-yl)propynyl]phenyl]allyloxy]-2-methyl-phenoxy]acetic acid (Compound 1) :

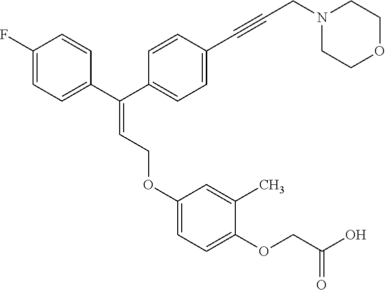 [SMILES: Cc1cc(OC/C=C(/c2ccc(F)cc2)c2ccc(C#CCN3CCOCC3)cc2)ccc1OCC(=O)O]

An example of the chemical synthesis of (E)-[4-[3-(4-Fluorophenyl)-3-[4-[3-(morpholin-4-yl)propynyl]phenyl]allyloxy]-2-methyl-phenoxy]acetic acid may be found in Example 10 of PCT Application Pub. No. WO 2007/071766.

Compound 1 was tested on all three human PPAR subtypes (hPPAR): hPPARα, hPPARγ, and hPPARδ in vitro assays testing for such activity. Compound 1 exhibited a significantly greater selectivity for PPARδ over PPARα and PPARγ (by at least about 100-fold and at least about 400-fold, respectively). In some cases, Compound 1 acts as a full agonist of PPARδ and only a partial agonist for both PPARα and PPARγ. In some cases, Compound 1 demonstrates negligible activity on PPARα and/or PPARγ in tranasctivation assays testing for such activity.

In some embodiments, Compound 1 did not show any human retinoid X receptor (hRXR) activity, or activity on the nuclear receptors FXR, LXR_{α} or LXR_{β}. as a full agonist of PPARδ and only a partial agonist for both PPARδ and PPARγ.

In some embodiments, a PPARδ agonist is (Z)-[2-Methyl-4-[3-(4-methylphenyl)-3-[4-[3-(morpholin-4-yl)propynyl]phenyl]allyloxy]-phenoxy]acetic acid:

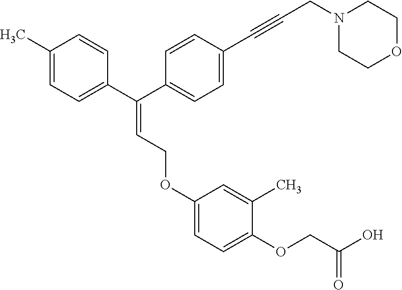 [SMILES: Cc1ccc(/C(=C/COc2ccc(OCC(=O)O)c(C)c2)c2ccc(C#CCN3CCOCC3)cc2)cc1]

An example of the chemical synthesis of (Z)-[2-Methyl-4-[3-(4-methylphenyl)-3-[4-[3-(morpholin-4-yl)propynyl]phenyl]allyloxy]-phenoxy]acetic acid may be found in Example 3 of PCT Application Pub. No. WO 2007/071766.

In some embodiments, a PPARδ agonist is (E)-[2-Methyl-4-[3-[4-[3-(pyrazol-1-yl)prop-1-ynyl]phenyl]-3-(4-trifluoromethylphenyl)-allyloxy]phenoxy]acetic acid:

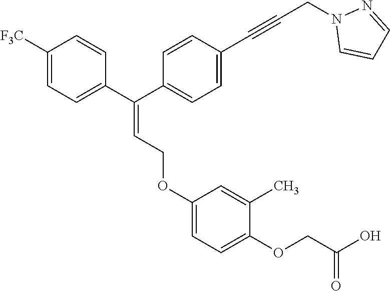 [SMILES: Cc1cc(OC/C=C(\c2ccc(C#CCn3cccn3)cc2)c2ccc(C(F)(F)F)cc2)ccc1OCC(=O)O]

An example of the chemical synthesis of (E)-[2-Methyl-4-[3-[4-[3-(pyrazol-1-yl)prop-1-ynyl]phenyl]-3-(4-trifluoromethylphenyl)-allyloxy]phenoxy]acetic acid may be found in Example 4 of PCT Application Pub. No. WO 2007/071766.

In some embodiments, a PPARδ agonist is (E)-[2-Methyl-4-[3-[4-[3-(morpholin-4-yl)propynyl]phenyl]-3-(4-trifluoromethylphenyl)allyloxy]-phenoxy]acetic acid:

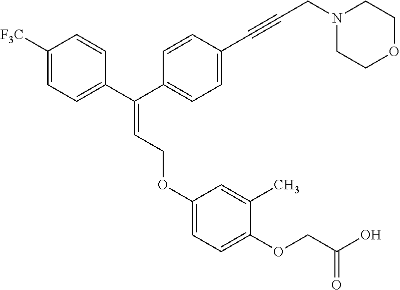 [SMILES: Cc1cc(OC/C=C(\c2ccc(C#CCN3CCOCC3)cc2)c2ccc(C(F)(F)F)cc2)ccc1OCC(=O)O]

An example of the chemical synthesis of (E)-[2-Methyl-4-[3-[4-[3-(morpholin-4-yl)propynyl]phenyl]-3-(4-trifluoromethylphenyl)allyloxy]-phenoxy]acetic acid may be found in Example 20 of PCT Application Pub. No. WO 2007/071766.

In some embodiments, a PPARδ agonist is (E)-[4-[3-(4-Chlorophenyl)-3-[4-[3-(morpholin-4-yl)propynyl]phenyl]allyloxy]-2-methyl-phenoxy]acetic acid:

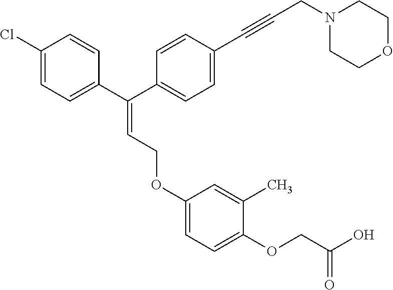 [SMILES: Cc1cc(OC/C=C(/c2ccc(Cl)cc2)c2ccc(C#CCN3CCOCC3)cc2)ccc1OCC(=O)O]

An example of the chemical synthesis of (E)-[4-[3-(4-Chlorophenyl)-3-[4-[3-(morpholin-4-yl)propynyl]phenyl]allyloxy]-2-methyl-phenoxy]acetic acid may be found in Example 46 of PCT Application Pub. No. WO 2007/071766.

In some embodiments, a PPARδ agonist is (E)-[4-[3-(4-Chlorophenyl)-3-[4-[3-(morpholin-4-yl)propynyl]phenyl]allyloxy]-2-methylphenyl]-propionic acid:

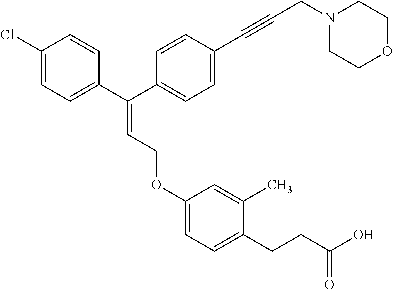 [SMILES: Cc1cc(OC/C=C(/c2ccc(Cl)cc2)c2ccc(C#CCN3CCOCC3)cc2)ccc1CCC(=O)O]

An example of the chemical synthesis of (E)-[4-[3-(4-Chlorophenyl)-3-[4-[3-(morpholin-4-yl)propynyl]phenyl]allyloxy]-2-methylphenyl]-propionic acid may be found in Example 63 of PCT Application Pub. No. WO 2007/071766.

In some embodiments, a PPARδ agonist is {4-[3-Isobutoxy-5-(3-morpholin-4-yl-prop-1-ynyl)-benzylsulfanyl]-2-methyl-phenoxy}-acetic acid:

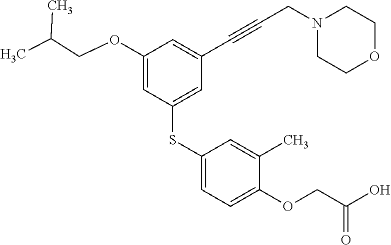 [SMILES: Cc1cc(Sc2cc(C#CCN3CCOCC3)cc(OCC(C)C)c2)ccc1OCC(=O)O]

An example of the chemical synthesis of {4-[3-Isobutoxy-5-(3-morpholin-4-yl-prop-1-ynyl)-benzylsulfanyl]-2-methyl-phenoxy}-acetic acid may be found in Example 9 of PCT Application Pub. No. WO 2007/003581.

In some embodiments, a PPARδ agonist is {4-[3-Isobutoxy-5-(3-morpholin-4-yl-prop-1-ynyl)-phenylsulfanyl]-2-methyl-phenoxy}-acetic acid:

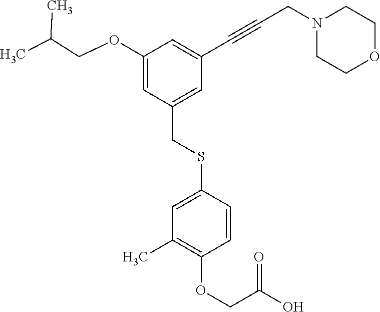 [SMILES: Cc1cc(SCc2cc(C#CCN3CCOCC3)cc(OCC(C)C)c2)ccc1OCC(=O)O]

An example of the chemical synthesis of {4-[3-Isobutoxy-5-(3-morpholin-4-yl-prop-1-ynyl)-phenylsulfanyl]-2-methyl-phenoxy}-acetic acid may be found in Example 35 of PCT Application Pub. No. WO 2007/003581.

In some embodiments, a PPARδ agonist is {4-[3,3-Bis-(4-bromo-phenyl)-allyloxy]-2-methyl-phenoxy}-acetic acid:

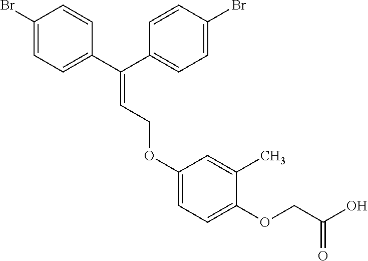 [SMILES: Cc1cc(OCC=C(c2ccc(Br)cc2)c2ccc(Br)cc2)ccc1OCC(=O)O]

An example of the chemical synthesis of {4-[3,3-Bis-(4-bromo-phenyl)-allyloxy]-2-methyl-phenoxy}-acetic acid may be found in Example 10 of PCT Application Pub. No. WO 2004/037776.

Accordingly, in an embodiment, a PPARδ agonist is a compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; and {4-[3,3-Bis-(4-bromo-phenyl)-allyloxy]-2-methyl-phenoxy}-acetic acid; or a pharmaceutically acceptable salt thereof.

In a further embodiment, a PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof. In some embodiments, the PPARδ agonist is (E)-[4-[3-(4-Fluorophenyl)-3-[4-[3-(morpholin-4-yl)propynyl]phenyl]allyloxy]-2-methyl-phenoxy]acetic acid sodium salt.

In a further embodiment, a PPARδ agonist is Comppound 1, Comppound 2, Comppound 3, Comppound 4, Comppound 5, Comppound 6, Comppound 7, Comppound 8, Comppound 9, Comppound 10, Comppound 11, Comppound 12, Comppound 13, Comppound 14, Comppound 15, or Comppound 16, disclosed in Wu et al. Proc Natl Acad Sci USA Mar. 28, 2017 114 (13) E2563-E2570.

In a further embodiment, a PPARδ agonist is (R)-3-methyl-6-(2-((5-methyl-2-(4-(trifluoromethyl)phenyl)-1H-imidazol-1-yl)methyl)phenoxy)hexanoic acid, or (R)-3-methyl-6-(2-((5-methyl-2-(6-(trifluoromethyl)pyridin-3-yl)-1H-imidazol-1-yl)methyl)phenoxy)hexanoic acid, or a pharmaceutically acceptable salt thereof.

In a further embodiment, a PPARδ agonist is (R)-3-methyl-6-(2-((5-methyl-2-(4-(trifluoromethyl)phenyl)-1H-imidazol-1-yl)methyl)phenoxy)hexanoic acid, or a pharmaceutically acceptable salt thereof. In some ebodiments, the PPARδ agonist is the hemisulfate salt of (R)-3-methyl-6-(2-((5-methyl-2-(4-(trifluoromethyl)phenyl)-1H-imidazol-1-yl)methyl)phenoxy)hexanoic acid. In some ebodiments, the PPARδ agonist is the meglumine salt of(R)-3-methyl-6-(2((5-methyl-2-(4-(trifluoromethyl)phenyl)-1H-imidazol-1-yl)methyl)phenoxy)hexanoic acid.

In a further embodiment, a PPARδ agonist is (R)-3-methyl-6-(2((5-methyl-2-(6-(trifluoromethyl)pyridin-3-yl)-1H-imidazol-1-yl)methyl)phenoxy)hexanoic acid, or a pharmaceutically acceptable salt thereof. In some ebodiments, the PPARδ agonist is the hemisulfate salt of (R)-3-methyl-6-(2-((5-methyl-2-(6-(trifluoromethyl)pyridin-3-yl)-1H-imidazol-1-yl)methyl)phenoxy)hexanoic acid. In some ebodiments, the PPARδ agonist is the meglumine salt of (R)-3-methyl-6-(2-((5-methyl-2-(6-(trifluoromethyppyridin-3-yl)-1H-imidazol-1-yl)methyl)phenoxy)hexanoic acid.

In a further embodiment, a PPARδ agonist is 2-(2-methyl-4-(((2-(4-(trifluoromethyl)phenyl)-2H-1,2,3-triazol-4-yl)methyl)thio)phenoxy)acetic acid, or a pharmaceutically acceptable salt thereof.

In a further embodiment, a PPARδ agonist is (R)-2-(4-((2-ethoxy-3-(4-(trifluoromethyl)phenoxy)propyl)thio)phenoxy)acetic acid, or a pharmaceutically acceptable salt thereof.

The term “pharmaceutically acceptable salt”in reference to a PPARδ agonist refers to a salt of the PPARδ agonist, which does not cause significant irritation to a mammal to which it is administered and does not substantially abrogate the biological activity and properties of the compound. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. In some embodiments, pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

In some embodiments, pharmaceutically acceptable salts are generally prepared by reacting the free base with a suitable organic or inorganic acid or by reacting the acid with a suitable organic or inorganic base. The term may be used in reference to any compound of the present invention. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isethionate, Lactate, Lactobionate, Laurate, Malate, Maleate, Mandelate, Mesylate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium, and Valerate. When an acidic substituent is present, such as —CO₂H, there can be formed the ammonium, morpholinium, sodium, potassium, barium, calcium salt, and the like for use as the dosage form. When a basic group is present, such as amino, or a basic heteroaryl radical, such as pyridyl, there can be formed an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate, and the like, and include acids related to the pharmaceutically acceptable salts listed in Stephen M. Berge, et al., Journal of Pharmaceutical Sciences, Vol. 66(1), pp. 1-19 (1977).

### Certain Terminology

Unless otherwise stated, the following terms used in this application have the definitions given below. The use of the term “including”as well as other forms, such as “include”, “includes,”and “included,”is not limiting. The section headings used herein are for organizational purposes only and are not to be construed as limiting the subject matter described.

The term “acceptable”with respect to a formulation, composition or ingredient, as used herein, means having no persistent detrimental effect on the general health of the subject being treated.

The term “modulate”as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator”as used herein, refers to a molecule that interacts with a target either directly or indirectly. The interactions include, but are not limited to, the interactions of an agonist, partial agonist, an inverse agonist, antagonist, degrader, or combinations thereof In some embodiments, a modulator is an antagonist. In some embodiments, a modulator is a degrader.

The terms “administer,”“administering”, “administration,”and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration”or the like, as used herein, are meant to encompass administration of the selected therapeutic agents to a single patient, and are intended to include treatment regimens in which the agents are administered by the same or different route of administration or at the same or different time.

The terms “effective amount”or “therapeutically effective amount,”as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount”for therapeutic uses is the amount of the composition comprising a compound as disclosed herein required to provide a clinically significant decrease in disease symptoms. An appropriate “effective”amount in any individual case is optionally determined using techniques, such as a dose escalation study.

The terms “enhance”or “enhancing,”as used herein, means to increase or prolong either in potency or duration a desired effect. Thus, in regard to enhancing the effect of therapeutic agents, the term “enhancing”refers to the ability to increase or prolong, either in potency or duration, the effect of other therapeutic agents on a system. An “enhancing-effective amount,”as used herein, refers to an amount adequate to enhance the effect of another therapeutic agent in a desired system.

The term “pharmaceutical combination”as used herein, means a product that results from the mixing or combining of more than one active ingredient and includes both fixed and non-fixed combinations of the active ingredients. The term “fixed combination”means that the active ingredients, e.g. a compound described herein, or a pharmaceutically acceptable salt thereof, and a co-agent, are both administered to a patient simultaneously in the form of a single entity or dosage. The term “non-fixed combination”means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit”and “article of manufacture”are used as synonyms.

The term “subject”or “patient”encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,”“treating”or “treatment,”as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remingtons Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments of the invention, a PPARδ agonist is included within a pharmaceutical composition. As used herein, the term “pharmaceutical composition”refers to a liquid or solid composition, preferably solid (e.g., a granulated powder), that contains a pharmaceutically active ingredient (e.g., a PPARδ agonist) and at least a carrier, where none of the ingredients is generally biologically undesirable at the administered quantities.

Pharmaceutical compositions incorporating a PPARδ agonist may take any physical form that is pharmaceutically acceptable. Pharmaceutical compositions for oral administration are particularly preferred. In one embodiment of such pharmaceutical compositions, an effective amount of a PPARδ agonist is incorporated.

The inert ingredients and manner of formulation of the pharmaceutical compositions of the invention are conventional. Known methods of formulation used in pharmaceutical science may be followed. All of the usual types of compositions are contemplated, including, but not limited to, tablets, chewable tablets, capsules, and solutions. The amount of the PPARδ agonist, however, is best defined as the effective amount, that is, the amount of the PPARδ agonist that provides the desired dose to the subject in need of such treatment. The activity of the PPARδ agonists does not depend on the nature of the composition, so the compositions may be chosen and formulated solely for convenience and economy. Any of the PPARδ agonists as described herein may be formulated in any desired form of composition.

Capsules may be prepared by mixing the PPARδ agonist with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets may be prepared by direct compression, by wet granulation, or by dry granulation. Their formulations usually incorporate diluents, binders, lubricants, and disintegrators, as well as the PPARδ agonist. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders are substances such as starch, gelatin, and sugars such as lactose, fructose, glucose, and the like. Natural and synthetic gums are also convenient, including acacia, alginates, methylcellulose, polyvinylpyrrolidine, and the like. Polyethylene glycol, ethylcellulose, and waxes can also serve as binders.

A lubricant in a tablet formulation may help prevent the tablet and punches from sticking in the die. A lubricant can be chosen from such solids as talc, magnesium and calcium stearate, stearic acid, and hydrogenated vegetable oils.

Tablet disintegrators are substances that swell when wetted to break up the tablet and release the compound. They include starches, clays, celluloses, aligns, and gums. More particularly, corn and potato starches, methylcellulose, agar, bentonite, wood cellulose, powdered natural sponge, cation-exchange resins, alginic acid, guar gum, citrus pulp, and carboxymethylcellulose, for example, may be used, as well as sodium lauryl sulfate.

Enteric formulations are often used to protect an active ingredient from the strongly acidic contents of the stomach. Such formulations are created by coating a solid dosage form with a film of a polymer that is insoluble in acid environments, and soluble in basic environments. Exemplary films are cellulose acetate phthalate, polyvinyl acetate phthalate, hydroxypropyl methylcellulose phthalate, and hydroxypropyl methylcellulose acetate succinate.

Tablets are often coated with sugar as a flavor and sealant. The PPARδ agonists may also be formulated as chewable tablets by using large amounts of pleasant-tasting substances such as mannitol in the formulation, as is now well-established practice.

Transdermal patches may be used. Typically, a patch comprises a resinous composition in which the active compound(s) will dissolve, or partially dissolve, and is held in contact with the skin by a film that protects the composition. Other, more complicated patch compositions are also in use, particularly those having a membrane pierced with innumerable pores through which the drugs are pumped by osmotic action.

In any embodiment where a PPARδ agonist is included in a pharmaceutical composition, such pharmaceutical compositions may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate, or sodium phosphate; granulating and disintegrating agents, for example, corn starch or alginic acid; binding agents, for example, starch, gelatin, or acacia; and lubricating agents, for example, magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl di stearate may be employed.

### Methods of Dosing and Treatment Regimens

In one embodiment, a PPARδ agonist (e.g. Compound 1, or a pharmaceutically acceptable salt thereof), is used in the preparation of medicaments for the treatment of kidney diseases or conditions. Methods for treating any of the diseases or conditions described herein in a mammal in need of such treatment, involves administration of pharmaceutical compositions that include a PPARδ agonist (e.g. Compound 1, or a pharmaceutically acceptable salt thereof), active metabolite, prodrug, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patients health status, weight, and response to the drugs, and the judgment of the treating physician. Therapeutically effective amounts are optionally determined by methods including, but not limited to, a dose escalation and/or dose ranging clinical trial.

In prophylactic applications, compositions containing a PPARδ agonist (e.g. Compound 1, or a pharmaceutically acceptable salt thereof), are administered to a patient susceptible to or otherwise at risk of a particular disease, disorder or condition. Such an amount is defined to be a “prophylactically effective amount or dose.”In this use, the precise amounts also depend on the patients state of health, weight, and the like. When used in patients, effective amounts for this use will depend on the severity and course of the disease, disorder or condition, previous therapy, the patients health status and response to the drugs, and the judgment of the treating physician. In one aspect, prophylactic treatments include administering to a mammal, who previously experienced at least one symptom of the disease being treated and is currently in remission, a pharmaceutical composition comprising a PPARδ agonist (e.g. Compound 1, or a pharmaceutically acceptable salt thereof), in order to prevent a return of the symptoms of the disease or condition.

In certain embodiments wherein the patients condition does not improve, upon the doctors discretion the administration of a PPARδ agonist (e.g. Compound 1, or a pharmaceutically acceptable salt thereof), is administered chronically, that is, for an extended period of time, including throughout the duration of the patients life in order to ameliorate or otherwise control or limit the symptoms of the patients disease or condition.

In certain embodiments wherein a patients status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patients conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

In one aspect, a PPARδ agonist (e.g. Compound 1, or a pharmaceutically acceptable salt thereof), is administered daily to humans in need of therapy a PPARδ agonist (e.g. Compound 1, or a pharmaceutically acceptable salt thereof). In some embodiments, a PPARδ agonist (e.g. Compound 1, or a pharmaceutically acceptable salt thereof), is administered once-a-day. In some embodiments, a PPARδ agonist (e.g. Compound 1, or a pharmaceutically acceptable salt thereof), is administered twice-a-day. In some embodiments, a PPARδ agonist (e.g. Compound 1, or a pharmaceutically acceptable salt thereof), is administered three times-a-day. In some embodiments, a PPARδ agonist (e.g. Compound 1, or a pharmaceutically acceptable salt thereof), is administered every other day. In some embodiments, a PPARδ agonist (e.g. Compound 1, or a pharmaceutically acceptable salt thereof), is administered twice a week.

In general, doses of a PPARδ agonist (e.g. Compound 1, or a pharmaceutically acceptable salt thereof), employed for treatment of the diseases or conditions described herein in humans are typically in the range of from about 0.1 mg to about 10 mg/kg of body weight per dose. In one embodiment, the desired dose is conveniently presented in a single dose or in divided doses administered simultaneously (or over a short period of time) or at appropriate intervals, for example as two, three, four or more sub-doses per day. In some embodiments, a PPARδ agonist (e.g. Compound 1, or a pharmaceutically acceptable salt thereof), is conveniently presented in divided doses that are administered simultaneously (or over a short period of time) once a day. In some embodiments, a PPARδ agonist (e.g. Compound 1, or a pharmaceutically acceptable salt thereof), is conveniently presented in divided doses that are administered in equal portions twice-a-day.

In some embodiments, a PPARδ agonist (e.g. Compound 1, or a pharmaceutically acceptable salt thereof), is administered orally to the human at a dose from about 0.1 mg to about 10 mg/kg of body weigh per dose. In some embodiments, a PPARδ agonist (e.g. Compound 1, or a pharmaceutically acceptable salt thereof), is administered to the human on a continuous daily dosing schedule.

The term “continuous dosing schedule”refers to the administration of a particular therapeutic agent at regular intervals. In some embodiments, continuous dosing schedule refers to the administration of a particular therapeutic agent at regular intervals without any drug holidays from the particular therapeutic agent. In some other embodiments, continuous dosing schedule refers to the administration of a particular therapeutic agent in cycles. In some other embodiments, continuous dosing schedule refers to the administration of a particular therapeutic agent in cycles of drug administration followed by a drug holiday (for example, a wash out period or other such period of time when the drug is not administered) from the particular therapeutic agent. For example, in some embodiments the therapeutic agent is administered once a day, twice a day, three times a day, once a week, twice a week, three times a week, four times a week, five times a week, six times a week, seven times a week, every other day, every third day, every fourth day, daily for a week followed by a week of no administration of the therapeutic agent, daily for a two weeks followed by one or two weeks of no administration of the therapeutic agent, daily for three weeks followed by one, two or three weeks of no administration of the therapeutic agent, daily for four weeks followed by one, two, three or four weeks of no administration of the therapeutic agent, weekly administration of the therapeutic agent followed by a week of no administration of the therapeutic agent, or biweekly administration of the therapeutic agent followed by two weeks of no administration of the therapeutic agent. In some embodiments, daily administration is once a day. In some embodiments, daily administration is twice a day. In some embodiments, daily administration is three times a day. In some embodiments, daily administration is more than three times a day.

The term “continuous daily dosing schedule”refers to the administration of a particular therapeutic agent everyday at roughly the same time each day. In some embodiments, daily administration is once a day. In some embodiments, daily administration is twice a day. In some embodiments, daily administration is three times a day. In some embodiments, daily administration is more than three times a day.

In some embodiments, the amount of a PPARδ agonist (e.g. Compound 1, or a pharmaceutically acceptable salt thereof), is administered once-a-day. In some other embodiments, the amount of a PPARδ agonist (e.g. Compound 1, or a pharmaceutically acceptable salt thereof), is administered twice-a-day. In some other embodiments, the amount of a PPARδ agonist (e.g. Compound 1, or a pharmaceutically acceptable salt thereof), is administered three times a day.

In certain embodiments wherein improvement in the status of the disease or condition in the human is not observed, the daily dose of a PPARδ agonist (e.g. Compound 1, or a pharmaceutically acceptable salt thereof), is increased. In some embodiments, a once-a-day dosing schedule is changed to a twice-a-day dosing schedule. In some embodiments, a three times a day dosing schedule is employed to increase the amount of a PPARδ agonist (e.g. Compound 1, or a pharmaceutically acceptable salt thereof), that is administered. In some embodiments, the frequency of administration by inhalation is increased in order to provide repeat high Cmax levels on a more regular basis. In some embodiments, the frequency of administration is increased in order to provide maintained or more regular exposure to a PPARδ agonist (e.g. Compound 1, or a pharmaceutically acceptable salt thereof). In some embodiments, the frequency of administration is increased in order to provide repeat high Cmax levels on a more regular basis and provide maintained or more regular exposure to a PPARδ agonist (e.g. Compound 1, or a pharmaceutically acceptable salt thereof).

In any of the aforementioned aspects are further embodiments comprising single administrations of the effective amount of a PPARδ agonist (e.g. Compound 1, or a pharmaceutically acceptable salt thereof), including further embodiments in which the PPARδ agonist, is administered (i) once a day; or (ii) multiple times over the span of one day.

In any of the aforementioned aspects are further embodiments comprising multiple administrations of the effective amount of a PPARδ agonist (e.g. Compound 1, or a pharmaceutically acceptable salt thereof), including further embodiments in which (i) the PPARδ agonist is administered continuously or intermittently: as in a single dose; (ii) the time between multiple administrations is every 6 hours; (iii) the PPARδ agonist is administered to the mammal every 8 hours; (iv) the PPARδ agonist is administered to the mammal every 12 hours; (v) the PPARδ agonist is administered to the mammal every 24 hours. In further or alternative embodiments, the method comprises a drug holiday, wherein the administration of the PPARδ agonist is temporarily suspended or the dose of the PPARδ agonist being administered is temporarily reduced; at the end of the drug holiday, dosing of the PPARδ agonist is resumed. In one embodiment, the length of the drug holiday varies from 2 days to 1 year.

Generally, a suitable dose of a PPARδ agonist, or a pharmaceutically acceptable salt thereof, for administration to a human will be in the range of about 0.1 mg/kg per day to about 25 mg/kg per day (e.g., about 0.2 mg/kg per day, about 0.3 mg/kg per day, about 0.4 mg/kg per day, about 0.5 mg/kg per day, about 0.6 mg/kg per day, about 0.7 mg/kg per day, about 0.8 mg/kg per day, about 0.9 mg/kg per day, about 1 mg/kg per day, about 2 mg/kg per day, about 3 mg/kg per day, about 4 mg/kg per day, about 5 mg/kg per day, about 6 mg/kg per day, about 7 mg/kg per day, about 8 mg/kg per day, about 9 mg/kg per day, about 10 mg/kg per day, about 15 mg/kg per day, about 20 mg/kg per day, or about 25 mg/kg per day). Alternatively, a suitable dose of a PPARδ agonist, or a pharmaceutically acceptable salt thereof, for administration to a human will be in the range of from about 0.1 mg/day to about 1000 mg/day; from about 1 mg/day to about 400 mg/day; or from about 1 mg/day to about 300 mg/day. In other embodiments, a suitable dose of a PPARδ agonist, or a pharmaceutically acceptable salt thereof, for administration to a human will be about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, or about 500 mg/day. Dosages may be administered more than one time per day (e.g., two, three, four, or more times per day). In one embodiment, a suitable dose of a PPARδ agonist, or a pharmaceutically acceptable salt thereof, for administration to a human is about 100 mg twice/day (i.e., a total of about 200 mg/day). In another embodiment, a suitable dose of a PPARδ agonist, or a pharmaceutically acceptable salt thereof, for administration to a human is about 50 mg twice/day (i.e., a total of about 100 mg/day).

In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the disease or condition to be treated, the mode of administration, the requirements of the individual subject, the severity of the disease or condition being treated, the identity (e.g., weight) of the human, and the particular additional therapeutic agents that are administered (if applicable), and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the PPARδ agonist lies within a range of circulating concentrations that include the ED₅₀ with minimal toxicity. In certain embodiments, the daily dosage range and/or the unit dosage amount varies within this range depending upon the dosage form employed and the route of administration utilized.

In some embodiments, following the administration of a therapeutically effective dose of the PPARδ agonist to a subject, the no observed adverse effect level (NOAEL) is at least 1, 10, 20, 50, 100, 500 or 1000 milligrams of the PPARδ agonist per kilogram of body weight (mpk). In some examples, the 7-day NOAEL for a rat administered PPARδ agonist is at least about 200, 300, 400, 500, 600, 700, 800, 900, 1000, 1500 or 2000 mpk. In some examples, the 7-day NOAEL for a dog administered PPARδ agonist is at least about 10, 20, 30, 40, 50, 60, 70, 80, 90, 100, 200, 500 mpk.

### Combination Treatments

In certain instances, it is appropriate to administer a PPARδ agonist (e.g. Compound 1, or a pharmaceutically acceptable salt thereof), in combination with one or more other therapeutic agents.

In one embodiment, the therapeutic effectiveness of a PPARδ agonist (e.g. Compound 1), or a pharmaceutically acceptable salt or solvate thereof, is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering a PPARδ agonist (e.g. Compound 1) , or a pharmaceutically acceptable salt or solvate thereof, with another agent (which also includes a therapeutic regimen) that also has therapeutic benefit.

In one specific embodiment, a PPARδ agonist (e.g. Compound 1) , or a pharmaceutically acceptable salt or solvate thereof, is co-administered with a second therapeutic agent, wherein a PPARδ agonist (e.g. Compound 1) , or a pharmaceutically acceptable salt or solvate thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

In certain embodiments, different therapeutically-effective dosages of a PPARδ agonist (e.g. Compound 1) , or a pharmaceutically acceptable salt or solvate thereof, will be utilized in formulating pharmaceutical composition and/or in treatment regimens when a PPARδ agonist (e.g. Compound 1) , or a pharmaceutically acceptable salt or solvate thereof, is administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a PPARδ agonist (e.g. Compound 1) , or a pharmaceutically acceptable salt or solvate thereof, is initiated prior to, during, or after treatment with a second agent described herein, and continues until any time during treatment with the second agent or after termination of treatment with the second agent. It also includes treatments in which a PPARδ agonist (e.g. Compound 1) , or a pharmaceutically acceptable salt or solvate thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, a PPARδ agonist (e.g. Compound 1) , or a pharmaceutically acceptable salt or solvate thereof, is administered either simultaneously with the one or more other therapeutic agents, or sequentially.

In combination therapies, the multiple therapeutic agents (one of which is a PPARδ agonist (e.g. Compound 1) , or a pharmaceutically acceptable salt or solvate thereof) are administered in any order or even simultaneously. If administration is simultaneous, the multiple therapeutic agents are, by way of example only, provided in a single, unified form, or in multiple forms (e.g., as a single pill or as two separate pills).

A PPARδ agonist (e.g. Compound 1) , or a pharmaceutically acceptable salt or solvate thereof, as well as combination therapies, are administered before, during or after the occurrence of a disease or condition, and the timing of administering the composition containing a PPARδ agonist (e.g. Compound 1) , or a pharmaceutically acceptable salt or solvate thereof, varies. Thus, in one embodiment, Compound I, or a pharmaceutically acceptable salt or solvate thereof, is used as a prophylactic and are administered continuously to subjects with a propensity to develop conditions or diseases in order to prevent the occurrence of the disease or condition. In another embodiment, a PPARδ agonist (e.g. Compound 1) , or a pharmaceutically acceptable salt or solvate thereof, is administered to a subject during or as soon as possible after the onset of the symptoms. In specific embodiments, a PPARδ agonist (e.g. Compound 1) , or a pharmaceutically acceptable salt or solvate thereof, is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a PPARδ agonist (e.g. Compound 1) , or a pharmaceutically acceptable salt or solvate thereof, or a formulation containing Compound I, or a pharmaceutically acceptable salt or solvate thereof, is administered for at least 2 weeks, about 1 month to about 5 years.

### Exemplary Agents for Use in Combination Therapy

In some embodiments, a PPARδ agonist (e.g. Compound 1, or a pharmaceutically acceptable salt), is administered in combination with a calcineurin inhibitor, a corticosteroid, a blocker of the renin-angiotensin-aldosterone system (RAAS), a diuretic, an angiotensin-converting enzyme (ACE) inhibition, angiotensin receptor blocker (ARB), inhibitors of TGF-β1, matrix metalloproteinases, vasopeptidase A or HMG-CoA reductase; chemokine receptor 1 blockade, BMP-7, stem cells, NAD+ modulator, irradiation, or combinations thereof.

In certain embodiments, the at least one additional therapy is administered at the same time as a PPARδ agonist (e.g. Compound 1) , or a pharmaceutically acceptable salt or solvate thereof In certain embodiments, the at least one additional therapy is administered less frequently than a PPARδ agonist (e.g. Compound 1) , or a pharmaceutically acceptable salt or solvate thereof In certain embodiments, the at least one additional therapy is administered more frequently than a PPARδ agonist (e.g. Compound 1) , or a pharmaceutically acceptable salt or solvate thereof. In certain embodiments, the at least one additional therapy is administered prior to administration of a PPARδ agonist (e.g. Compound 1) , or a pharmaceutically acceptable salt or solvate thereof. In certain embodiments, the at least one additional therapy is administered after administration of a PPARδ agonist (e.g. Compound 1) , or a pharmaceutically acceptable salt or solvate thereof.

Calcineurin inhibitors include, but are not limited to, cyclosporin, and tacrolimus.

Corticosteroids include, but are not limited to, betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Agents that interfere with RAAS include: angiotensin converting enzyme (ACE) inhibitors, angiotensin receptor blockers (ARBs), aldosterone inhibitors.

ACE inhibitors include, but are not limited to, benazepril, cilazapril, enalapril, fosinopril, lisinopril, perinopril, ramapril, quinapril, and trandolapril.

ARBs include, but are not limited to, candesartan, epresartan, irbesartan, losartan, telmisartan, and valsartan.

Aldosterone inhibitors include, but are not limited to, spironolactone.

In some embodiments, a PPARδ agonist is administered in combination with a Nicotinamide Adenine Dinucleotide (NAD+) pathway modulator. NAD+ plays many important roles within cells, including serving as an oxidizing agent in oxidative phosphorylation which generates ATP from ADP. Increasing cellular concentrations of NAD-+ will enhance the oxidative capacity within mitochondria, thereby increasing nutrient oxidation and boost energy supply, which is a primary role of mitochondria. In some embodimens the NAD+ modulator targets Poly ADP Ribose Polymerase (PARP), Aminocarboxymuconate Semialdehyde Decarboxylase (ACMSD) and N′-Nicotinamide Methyltransferase (NNMT).

The term “irradiation”or “radiotherapy”or “ionizing radiation”include all forms of radiation, including but not limited to α, β, and γ radiation and ultraviolet light.

In some embodiments, a PPARδ agonist is administered in combination with a treatment targeting proteinuria. Treatment targeting proteinuria include, but are not limited to, calcineurin inhibitors and blockers of the renin-angiotensin-aldosterone system (RAAS).

In some embodiments, a PPARδ agonist is administered in combination with a treatment targeting inflammation and fibrosis. Treatments targeting inflammation and fibrosis include, but are not limited to, Complement inhibition, chemokine receptor antagonists, bone morphogenetic protein-7 (BMP-7), matrix metalloproteinase inhibitors.

In some embodiments, a PPARδ agonist is administered in combination with a treatment targeting the GBM pathology of Alport syndrome. Treatments targeting the GBM pathology of Alport syndrome include, but are not limited to, Discoidin domain receptor 1 and integrin x2131 antagonism.

In some embodiments, a PPARδ agonist is administered in combination with endothelin receptor antagonists (antiproteinuric effect), 3-hydroxy-3-methylglutaryl CoA (HMG-CoA) reductase inhibitors (antihypertensive and antiproteinuric effect), vasopeptidase inhibitors (antiproteinuric and glomerular hemodynamic effects), pentoxifylline (methylxanthine derivative that downregulates TNF-α) and vitamin D (antiproteinuric, anti-inflammatory and immunomodulatory effects).

### EXAMPLES

The following examples are provided for illustrative purposes only and not to limit the scope of the claims provided herein.

### Example 1

### Mouse Alport Model of Kidney Fibrosis

Mice with mutations in one of the collagen IV genes of glomerular basement membrane collagen, Collagen IV-α3/α4/α5, have defects in glomerular function with development of kidney fibrosis. These mice develop renal dysfunction and die prematurely of renal failure with specific timing dependent on the strain background upon which the mutation is present.

### Material and Methods:

Generation of Col4a3^{-/-}mice was as described in Miner, J. H., and Sanes, J. R. (1996), J. Cell Biol. 135:1403-1413. Studies of Compound 1 efficacy in Alport models of kidney fibrosis were performed using mice on two different strain backgrounds. Study 1 used B 6129S1 F1 hybrid Alport mice that usually reach end stage renal disease (ESRD) at about 17 to 19 weeks. These were generated by crosses between C57BL6/J (B 6) and 129S1/Sv1mJ (129S1) Alport mice. Study 2 used inbred 129S1 Col4a3^{-/-} mice, which usually reach ESRD at about 11 to 13 weeks.

### Treatment:

Study 1—B6129S1 hybrid Col4a3^{-/-} mice were given administrations via intraperitoneal injection of either vehicle (PBS) or Compound 1 (10 mg/kg) once a day from 6 to 17-weeks of age. There were 12 mice per group. B6129S1 hybrid Col4a3^{+/-} mice were used as non-disease controls. Compound 1 was dissolved at 2 mg/mL in PBS twice a week. The dosing volume was calculated as body weight (g)*5 μL (e.g. body weight 20 g: 20*5=100 μL).

Study 2—Inbred 129S1 Col4a3^{-/-} mice were administered either vehicle or Compound 1 via intraperitoneal injection (3 or 10 mg/kg) once a day from 4 to 10-weeks of age. There were 12 mice per group. Compound 1 was dissolved at 0.6 and 2 mg/mL in PBS twice per week. The dosing volume was body weight (g)*5 μL (e.g. body weight 20 g: 20*5=100 μL)

### Samples and Analyses:

Urine and blood samples were collected at the time points of 12, 15, and 17-weeks of age for B6129S1 hybrid Col4a3^{+/-, -/-} mice and at 8 and 10-weeks of age for inbred 129S1 Col4a3^{-/-} mice. Urinary protein concentration was determined by Bradford assay (Cat. 5000006, Bio-Rad) and comparison with bovine serum albumin standards. Urinary creatinine was measured by QuantiChrom™ Creatinine Assay Kit (Cat.DICT-500, BioAssay systems) and used to normalize the amount of urine. Blood urea nitrogen (BUN) levels were measured by QuantiChrom™ Urea Assay Kit (Cat.DUR2-100, BioAssay systems). Urine and blood samples were stored at −20° C. and −80° C. respectively after collection and subsequently analyzed.

For biochemistry, kidneys were collected from 15 and 17-week old B 6129S1 hybridCol4a3^{+/-, -/-} mice; and from 8 and 10-week old inbred 129S1 Col4a3^{-/-} mice. Collected kidneys were quickly frozen by liquid nitrogen and stored at −80° C. Kidneys were minced in cold PBS containing protease inhibitor (Cat. 786-108, G-BIOSCIENCES), centrifuged, and supernatants were removed. Tissue pellets were washed with PBS and centrifuged again. Tissue pellets were lysed with RIPA buffer containing protease inhibitor and phosphatase inhibitor (Sigma-Aldrich, Cat. P5726). Protein concentration was measured by BCA assays (Cat. 23227, ThermoFisher). The following antibodies were used in western blotting: anti-kidney injury molecule 1 (KIM1) antibody (AF1817, R&D), anti-Lipocalin2/NGAL antibody (ab70287, abcam), anti-phosho-STAT3 antibody (#9145, CST), anti-CTGFantibody (sc-365970, Santa Cruz), anti-TGFb 1,2,3 antibody (MAB1835, R&D), anti-Fibronectin antibody (F3648,Sigma), anti-alpha smooth muscle acting (SMA) antibody (F3777, Sigma), anti-Collagen I antibody (1310-01, SouthernBiotech), anti-Collagen IV antibody (1340-01, SouthernBiotech), anti-alpha Tubulin antibody (#2144, CST).

For all groups, analysis includes measurement of blood urea nitrogen and urinary albumin: creatinine ratios. The analysis also includes renal histopathology (glomerulosclerosis and tubulo-interstitial fibrosis; and analysis of glomerular basement membrane and podocyte ultrastructure. Plasma samples are analyzed for Compound 1 concentration to ensure adequate circulating levels for efficient target engagement. qRT-PCR is performed using RNA from several relevant tissues to examine expression of known transcriptional targets of PPARδ (e.g., PGC1α).

### Statistics:

All data are presented as mean±SEM. Statistical significance was determined by Oneway ANOVA with Dunnett's multiple comparison for more than three group comparison and Student's t test for two group comparison. Statistical significance was showed as *p≤0.05,**p<0.01, ***p<0.005, ****p<0.001.

### Results: Study 1

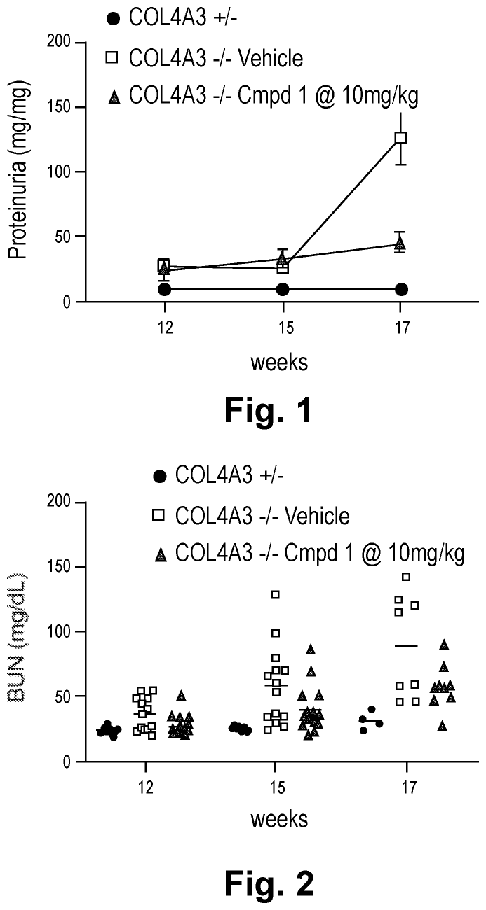

Compound 1 treatment attenuated kidney dysfunction in B6129S1 hybrid Col4a3^{-/-} mice. Compound 1 treatment suppressed proteinuria at 17-weeks of age, which is the late stage of kidney disease (FIG. 1). Compound 1 also suppressed the increase of blood urea nitrogen (BUN) at 12 and 17-weeks of age (FIG. 2).

### Results: Study 2

Compound 1 did not attenuate Kidney dysfunction in 129S1 Col4a3^{-/-} mice. The severity of Alport syndrome in mice is dependent on mouse strain background. The 129S1 strain is more severe than B6 strain (129S1 and B6 Col4a3^{-/-} reached ESRD at 80±7.8d and 114.1±14.1d (mean±SD) respectively) (Kang, J. S., et al., (2006), J Am Soc Nephrol, 17:1962-1969). To investigate the effect of Compound 1 on the more rapidly progressive Alport syndrome model, vehicle (PBS) or Compound 1 (3 mg/kg and 10 mg/kg) was administered via once daily intraperitoneal injections into 129S1 Col4a3^{-/-} mice from 4 to 10-weeks of age. During treatment, urine and blood samples were collected at 8 and 10-weeks of age for evaluating kidney function. In contrast to B6129S1 hybrid Col4a3^{-/-} mice in Study 1, the treatment of inbred 129S1 Col4a3^{-/-} mice with Compound 1 did not result in any significant reduction of proteinuria or BUN (data not shown).

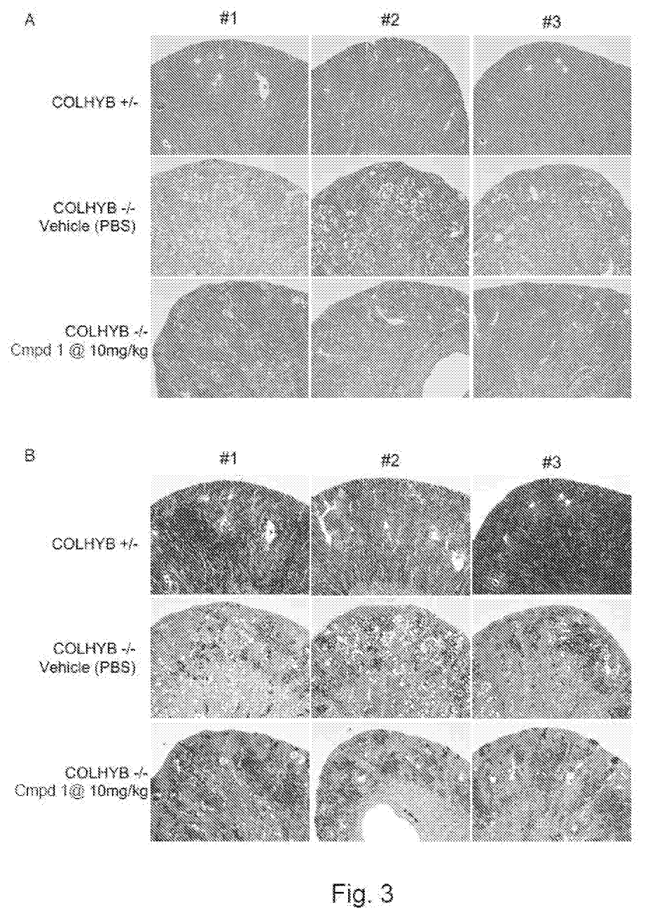

Compound 1 treatment slightly improved renal histology on B6129S hybrid Col4a3^{-/-} mice. Because Compound 1 had a protective effect at a late disease stage in Study 1, Compound 1 may have attenuated inflammation and fibrosis to improve late stage kidney disease. To analyze tissue inflammation and fibrosis, kidney sections were stained with H&E and Trichrome. Microscopic examination showed Compound 1 slightly reduced the infiltration of inflammatory cells. Also, necrotic regions in the cortex was decreased in Compound 1-treated B6129S1 hybrid Col4a3^{-/-} mice compared with vehicle-treated mice. (FIG. 3A). These results indicate that Compound 1 suppressed renal inflammation. In addition, the extent of fibrosis appeared slightly decreased in Compound 1 treated B 6129S1 hybrid Col4a3^{-/-} mice compared with vehicle-treated mice. (FIG. 3B). Thus, Compound 1 treatment in Study 1 down-regulated the expression of inflammatory and fibrosis-related molecules in whole kidneys.

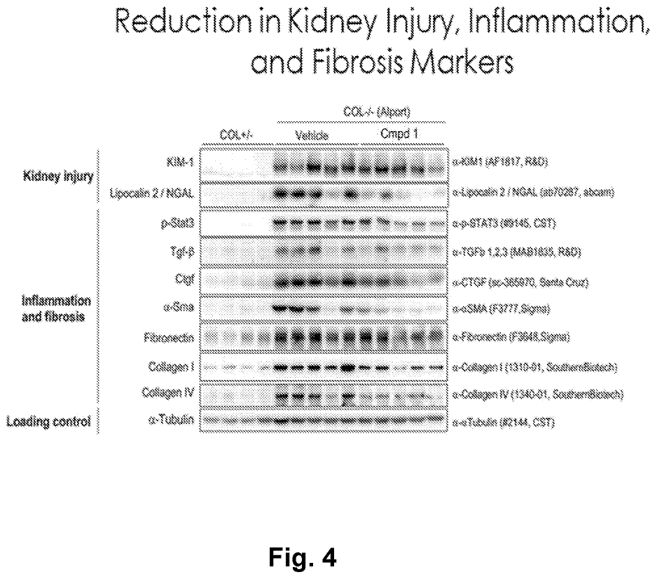

Compound 1 treatment in Study 1 down-regulated the expression of inflammatory and fibrosis-related molecules in whole kidneys. To further investigate the effect of Compound 1, the expression of molecules related to inflammation and fibrosis were evaluated by western blotting. Kidney injury molecule (KIM)-1 and Lipocalin-1/neutrophil gelatinase-associated lipocalin (NGAL) were elevated in nontreated B6129S1 hybrid Col4a3−/− mice compared with healthy control Col4a3+/−mice. NGAL protein level was decreased in Compound 1-treated B6129S1 hybrid Col4a3−/− mice. In contrast, KIM-1 protein level was not changed between vehicle and Compound 1 treated Col4a3−/−mice. The inflammation and fibrosis regulators phosho-Stat3, TGFβ, and connective tissue growth factor (CTGF) were upregulated in vehicle treated Col4a3^{-/-} mice, and phosho-Stat3 and CTGF expression were attenuated by Compound 1 treatment. Moreover, Compound 1 decreased expression of the activated fibroblast/myofibroblast marker alpha-SMA and of the extracellular matrix proteins Collagen I and IV, but fibronectin expression was not changed by Compound 1 treatment (FIG. 4). These results suggest that Compound 1 ameliorated kidney dysfunction by decreasing expression of specific proteins related to inflammation and fibrosis.

### Example 2

### Combination with Other Anti-Fibrotic Agents for Renal Fibrosis

PPARδ agonists can be used in combination with other drugs for chronic kidney diseases including Alport syndrome. Angiotensin II converting enzyme (ACE) inhibitors and angiotensin receptor blockers (ARBs) are frequently used in renal disease patients. PPARδ agonist are tested alone and in combination with ramipril (ACE inhibitor) or candesartan (ARB) in prophylactic dosing starting at 2-3 weeks of age. If efficacious after prophylactic dosing, combination studies in therapeutic dosing modality (starting 4-6 weeks of age) are performed. Fibrosis is measured using histologically as described above and renal function is measured using proteinuria and/or serum BUN. Effects of combination therapy on survival are also measured as described above. Combination therapy is advantageous when efficacy is greater than either agent alone or when the dose required for either drug is reduced thereby improving the side effect profile.

### Example 3

### Clinical Trial for Alport Syndrome

A non-limiting example of an Alport Syndrome clinical trial in humans is described below.

Purpose: The purposes of this study are to assess the efficacy of Compound 1, or a pharmaceutically acceptable salt thereof, as single agent or in combination, in the treatment of patients with Alport syndrome, collect information on any side effects the compound may cause as single agent or in combination, and evaluate the pharmacokinetic properties of the compound as single agent or in combination.

Intervention: Patients are administered 10-200 mg of Compound 1, or a pharmaceutically acceptable salt or solvate thereof, per day as single agent or in combination.

Detailed Description: Patients will be given Compound 1, or a pharmaceutically acceptable salt or solvate thereof, orally once or twice a day as single agent or in combination. Prior to each dosing cycle, a physical exam, blood work and assessment of any side effects will be performed.

Eligibility: 12 years to 60 years (child, adult).

Inclusion Criteria: Male and female patients 12≤age≤60 upon study consent; diagnosis of Alport syndrome by genetic testing (documented mutation in a gene associated with Alport syndrome, including COL4A3, COL4A4, or COL4A5) or histologic assessment using electron microscopy; Screening eGFR≥30 and ≤90 mL/min/1.73 m2; Albumin to creatinine ratio (ACR)≤3500 mg/g; If receiving an angiotensin-converting enzyme (ACE) inhibitor and/or an angiotensin II receptor blocker (ARB), the medications must remain the same for at least 6 weeks prior to participation in this study. Patients not taking an ACE inhibitor and/or ARB because of a medical contraindication must have discontinued treatment at least 8 weeks prior to participation; Adequate bone marrow reserve and organ function; Able to swallow capsules; Willing and able to comply with scheduled visits, treatment plan, laboratory tests, and other study procedures.

Exclusion Criteria: Prior exposure to Compound 1; Ongoing chronic hemodialysis or peritoneal dialysis therapy; Renal transplant recipient; B-type natriuretic peptide (BNP) level >200 pg/mL; Uncontrolled diabetes (HbAlc>11.0%); Acute dialysis or acute kidney injury within 12 weeks prior to participation; Serum albumin<3 g/dL; History of clinically significant left-sided heart disease and/or clinically significant cardiac disease, including but not limited to any of the following: Uncontrolled systemic hypertension as evidenced by sitting systolic blood pressure (BP)>160 mm Hg or sitting diastolic BP>100 mm Hg after a period of rest; Systolic BP<90 mm Hg after a period of rest; Systemic immunosuppression for more than 2 weeks, cumulatively, within the 12 weeks prior to randomization or anticipated need for immunosuppression during the study; Untreated or uncontrolled active bacterial, fungal, or viral infection; Participation in other interventional clinical studies within 30 days prior to Day 1; Unwilling to practice acceptable methods of birth control (both males who have partners of child-bearing potential and females of childbearing potential) during Screening, while taking study drug, and for at least 30 days after the last dose of study drug is ingested; Women who are pregnant or breastfeeding; Known hypersensitivity to any component of the study drug

Primary Outcome Measures: To assess the increase in eGFR (estimated glomerular filtration rate) from baseline to week 12-week 48 for patients receiving active drug, compared to patients receiving placebo.

Secondary Outcome Measures: To assess the change from baseline in eGFR in Compound 1-treated patients following a 4-week drug treatment withdrawal period.

The examples and embodiments described herein are for illustrative purposes only and various modifications or changes suggested to persons skilled in the art are to be included within the spirit and purview of this application and scope of the appended claims.

What is claimed is:

1. A method for treating kidney disease in a mammal, comprising administering to the mammal a peroxisome proliferator-activated receptor delta (PPARδ) agonist, wherein the mammal has one of more mutations in the genes encoding α3, α4, or α5 chains of collagen IV.

2. The method of claim 1, wherein:
the PPARδ agonist binds to and activates the cellular PPARδ and does not substantially activate the cellular peroxisome proliferator activated receptors—alpha (PPARα) and —gamma (PPARγ).

3. The method of claim 1 or claim 2, wherein:
the kidney disease is Alport syndrome, Goodpasture syndrome, thin basement membrane nephropathy (TBMN), focal segmental glomerulosclerosis (FSGS), benign familial hematuria (BFH), post-transplant anti-GBM (Glomerular Basement Membrane) nephritis

4. The method of claim 3, wherein:
the kidney disease is X-linked Alport syndrome (XLAS), autosomal recessive Alport syndrome (ARAS) or autosomal dominant Alport syndrome (ADAS).

5. The method of any one of claims 1-4, wherein:
the PPARδ agonist increases fatty acid oxidation (FAO) in kidney tissues, increases carnitine palmitoyl-transferase 1(CPT1) levels in kidney tissues, attenuates excessive collagen deposition in kidney tissues, increase mitochondrial function in kidney tissues, attenuate oxidative stress in kidney tissues, decrease inflammation in kidney tissues, or a combination thereof.

6. The method of any one of claims 1-5, wherein:
the method comprises reducing proteinuria, suppressing the increase of blood urea nitrogen (BUN), reducing intraglomerular pressure, ameliorating glomerular injury, ameliorating extracellular matrix deposition, reducing renal fibrosis, arresting a decline in the estimated glomerular filtration rate (eGFR), increasing eGFR, delaying the onset of end-stage renal disease (ESRD), or combinations thereof.

7. The method of any one of claims 1-6, wherein:
the method comprises achieving a urine protein:creatinine ratio of less than about 0.5 mg/mg if the baseline value is greater than about 1.0 mg/mg.

8. The method of any one of claims 1-6, wherein:
the method comprises achieving an about 50% reduction of urine protein:creatinine ratio if the baseline value is greater than about 0.2 but less than about 1.0.

9. The method of any one of claims 1-8, wherein:
the PPARδ agonist compound is a phenoxyalkylcarboxylic acid compound; or a pharmaceutically acceptable salt thereof.

10. The method of any one of claims 1-8, wherein:
the PPARδ agonist compound is a phenoxyethanoic acid compound, phenoxypropanoic acid compound, phenoxybutanoic acid compound, phenoxypentanoic acid compound, phenoxyhexanoic acid compound, phenoxyoctanoic acid compound, phenoxynonanoic acid compound, or phenoxydecanoic acid compound; or a pharmaceutically acceptable salt thereof.

11. The method of any one of claims 1-8, wherein:
the PPARδ agonist compound is a phenoxyethanoic acid compound or a phenoxyhexanoic acid compound; or a pharmaceutically acceptable salt thereof.

12. The method of any one of claims 1-8, wherein:
the PPARδ agonist compound is an allyloxyphenoxyethanoic acid acid compound; or a pharmaceutically acceptable salt thereof.

13. The method of any one of claims 1-8, wherein the PPARδ agonist compound is:
(E)-[4-[3-(4-Fluorophenyl)-3-[4-[3-(morpholin-4-yl)propynyl]phenyl]allyloxy]-2-methyl-phenoxy]acetic acid;
(Z)-[2-Methyl-4-[3-(4-methylphenyl)-3-[4-[3-(morpholin-4-yl)propynyl]phenyl]allyloxy]-phenoxy]acetic acid;
(E)-[2-Methyl-4-[3-[4-[3-(pyrazol-1-yl)prop-1-ynyl]phenyl]-3-(4-trifluoromethylphenyl)-allyloxy]phenoxy]acetic acid;
(E)-[2-Methyl-4-[3-[4-[3-(morpholin-4-yl)propynyl]phenyl]-3-(4-trifluoromethylphenyl)allyloxy]-phenoxy]acetic acid;
(E)-[4-[3-(4-Chlorophenyl)-3-[4-[3-(morpholin-4-yl)propynyl]phenyl]allyloxy]-2-methyl-phenoxy]acetic acid;
(E)-[4-[3-(4-Chlorophenyl)-3-[4-[3-(morpholin-4-yl)propynyl]phenyl]allyloxy]-2-methylphenyl]-propionic acid;
{4-[3-Isobutoxy-5-(3-morpholin-4-yl-prop-1-ynyl)-benzylsulfanyl]-2-methyl-phenoxy}-acetic acid;
{4-[3-Isobutoxy-5-(3-morpholin-4-yl-prop-1-ynyl)-phenylsulfanyl]-2-methyl-phenoxy}-acetic acid; or
{4-[3,3-Bis-(4-bromo-phenyl)-allyloxy]-2-methyl-phenoxy}-acetic acid;
or a pharmaceutically acceptable salt thereof.

14. The method of any one of claims 1-8, wherein the PPARδ agonist is:
(E)-[4-[3-(4-Fluorophenyl)-3-[4-[3-(morpholin-4-yl)propynyl]phenyl]allyloxy]-2-methyl-phenoxy]acetic acid;
(Z)-[2-Methyl-4-[3-(4-methylphenyl)-3-[4-[3-(morpholin-4-yl)propynyl]phenyl]allyloxy]-phenoxy]acetic acid;
(E)-[2-Methyl-4-[3-[4-[3-(pyrazol-1-yl)prop-1-ynyl]phenyl]-3-(4-trifluoromethylphenyl)-allyloxy]phenoxy]acetic acid;
(E)-[2-Methyl-4-[3-[4-[3-(morpholin-4-yl)propynyl]phenyl]-3-(4-trifluoromethylphenyl)allyloxy]-phenoxy]acetic acid;
(E)-[4-[3-(4-Chlorophenyl)-3-[4-[3-(morpholin-4-yl)propynyl]phenyl]allyl oxy]-2-methyl-phenoxy]aceti c acid;
(E)-[4-[3-(4-Chlorophenyl)-3-[4-[3-(morpholin-4-yl)propynyl]phenyl]allyloxy]-2-methylphenyl]-propionic acid;
{4-[3-Isobutoxy-5-(3-morpholin-4-yl-prop-1-ynyl)-benzylsulfanyl]-2-methyl-phenoxy}-acetic acid;
{4-[3-Isobutoxy-5-(3-morpholin-4-yl-prop-1-ynyl)-phenylsulfanyl]-2-methyl-phenoxy}-acetic acid;
{4-[3,3-Bis-(4-bromo-phenyl)-allyloxy]-2-methyl-phenoxy}-acetic acid;
(R)-3-methyl-6-(2-((5-methyl-2-(4-(trifluoromethyl)phenyl)-1H-imidazol-1-yl)methyl)phenoxy)hexanoic acid;
(R)-3-methyl-6-(2-((5-methyl-2-(6-(trifluoromethyl)pyridin-3-yl)-1H-imidazol-1-yl)methyl)phenoxy)hexanoic acid;
2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954);
2-[2-methyl-4-[[3-methyl-4-[[4-(trifluoromethyl)phenyl]methoxy]phenyl]thio]phenoxy]-acetic acid;
2-[2-methyl-4-[[[4-methyl-2-[4-(trifluoromethyl)phenyl]-5-thiazolyl]methyl]thio]phenoxy]-acetic acid (GW-501516);
[4-[[[2-[3-Fluoro-4-(trifluoromethyl)phenyl]-4-methyl-5-thiazolyl]methyl]thio]-2-methylphenoxy]acetic acid (GW0742 also known as GW610742);
2-[2,6 dimethyl-4-[3-[4-(methylthio)phenyl]-3-oxo-1(E)-propenyl]phenoxyl]-2-methylpropanoic acid (elafibranor; GFT-505);
{2-methyl-4-[5-methyl-2-(4-trifluoromethyl-phenyl)-2H-[1,2,3]triazol-4-ylmethylsulfanyl]-phenoxy}-acetic acid;
[4-({(2R)-2-Ethoxy-3-[4-(trifluoromethyl)phenoxy]propyl}sulfanyl)-2-methylphenoxy]acetic acid (seladelpar; MBX-8025);
(S)-4-[cis-2,6-dimethyl-4-(4-trifluoromethoxy-phenyl)piperazine-1-sulfonyl]-indan-2-carboxylic acid or a tosylate salt thereof (KD-3010);
(2s)-2-{4-butoxy-3-[({[2-Fluoro-4-(Trifluoromethyl)phenyl]carbonyl}amino)methyl]benzyl}butanoic acid (TIPP-204);
[4-[3-(4-Acetyl-3-hydroxy-2-propylphenoxy)propoxy]phenoxy]acetic acid (L-165,0411);
2-(4-{2-[(4-Chlorobenzoyl)amino]ethyl}phenoxy)-2-methylpropanoic acid (bezafibrate);
2-(2-methyl-4-(((2-(4-(trifluoromethyl)phenyl)-2H-1,2,3-triazol-4-yl)methyl)thio)phenoxy)acetic acid; or
(R)-2-(4-((2-ethoxy-3-(4-(trifluoromethyl)phenoxy)propyl)thio)phenoxy)acetic acid; or a pharmaceutically acceptable salt thereof.

15. The method of any one of claims 1-8, wherein:
the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof.

16. The method of any one of claims 1-8, wherein:
the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 10 mg to about 500 mg.

17. The method of any one of claims 1-8, wherein:
the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg to about 200 mg.

18. The method of any one of claims 1-8, wherein:
the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 75 mg to about 125 mg.

19. The method of any one of claims 1-18, wherein:
the PPARδ agonist is systemically administered to the mammal.

20. The method of any one of claims 1-19, wherein:
the PPARδ agonist is administered to the mammal orally, by injection or intraveneously.

21. The method of claim 20, wherein:
the PPARδ agonist is administered to the mammal in the form of an oral solution, oral suspension, powder, pill, tablet or capsule.

22. The method of any one of claims 1-21, further comprising:
administering at least one additional therapeutic to the mammal.

23. The method of claim 22, wherein:
the at least one additional therapeutic agent is a Nicotinamide Adenine Dinucleotide (NAD+) pathway modulator.

24. The method of claim 22, wherein:
the at least one additional therapeutic agent is a Poly ADP Ribose Polymerase (PARP) modulator, Aminocarboxymuconate Semialdehyde Decarboxylase (ACMSD) modulator or N′-Nicotinamide Methyltransferase (NNMT) modulator.

25. The method of claim 22, wherein:
the at least one additional therapeutic agent is an inhibitor of the renin-angiotensin-aldosterone system (RAAS).

26. The method of claim 22, wherein:
the at least one additional therapeutic agent is an angiotensin-converting enzyme (ACE) inhibitor, angiotensin-receptor blocker (ARB), aldosterone inhibitor, calcineurin inhibitor, TGF-β1 inhibitor, matrix metalloproteinase inhibitor, vasopeptidase A inhibitor or HMG-CoA reductase inhibitor, chemokine receptor 1 blocker.

27. The method of claim 26, wherein:
the angiotensin converting enzyme (ACE) inhibitor is benazepril, cilazapril, enalapril, fosinopril, lisinopril, perinopril, ramapril, quinapril, or trandolapril;
the angiotensin-receptor blocker (ARB) is candesartan, epresartan, irbesartan, losartan, telmisartan, or valsartan;
the aldosterone inhibitor is spironolactone.

28. The method of any one of claims 1-27, wherein:
the mammal is a human.

29. A method for increasing fatty acid oxidation (FAO), increasing carnitine palmitoyl-transferase 1(CPT1) levels, attenuating excessive collagen deposition, increasing mitochondrial function, increasing mitochondrial biogenesis, attenuating oxidative stress, decreasing inflammation, or a combination thereof, in the kidneys of a mammal with kidney disease, comprising administering a peroxisome proliferator-activated receptor delta (PPARδ) agonist to the mammal.

30. The method of claim 29, wherein:
the PPARδ agonist binds to and activates the cellular PPARδ and does not substantially activate the cellular peroxisome proliferator activated receptors—alpha (PPARα) and—gamma (PPARγ).

31. The method of claim 29 or claim 30, wherein:
the mammal has one of more mutations in the genes encoding α3, α4, or α5 chains of collagen IV.

32. The method of any one of claims 29-31, wherein:
the kidney disease is Alport syndrome, Goodpasture syndrome, thin basement membrane nephropathy (TBMN), focal segmental glomerulosclerosis (FSGS), benign familial hematuria (BFH), post-transplant anti-GBM (Glomerular Basement Membrane) nephritis.

33. The method of claim 32, wherein:
the kidney disease is X-linked Alport syndrome (XLAS), autosomal recessive Alport syndrome (ARAS) or autosomal dominant Alport syndrome (ADAS).

34. The method of any one of claims 29-33, wherein:
the PPARδ agonist compound is a phenoxyalkylcarboxylic acid compound; or a pharmaceutically acceptable salt thereof.

35. The method of any one of claims 29-33, wherein:
the PPARδ agonist compound is a phenoxyethanoic acid compound, phenoxypropanoic acid
compound, phenoxybutanoic acid compound, phenoxypentanoic acid compound, phenoxyhexanoic acid compound, phenoxyoctanoic acid compound, phenoxynonanoic acid compound, or phenoxydecanoic acid compound; or a pharmaceutically acceptable salt thereof.

36. The method of any one of claims 29-33, wherein:
the PPARδ agonist compound is a phenoxyethanoic acid compound or a phenoxyhexanoic acid compound; or a pharmaceutically acceptable salt thereof.

37. The method of any one of claims 29-33, wherein:
the PPARδ agonist compound is an allyloxyphenoxyethanoic acid acid compound; or a pharmaceutically acceptable salt thereof.

38. The method of any one of claims 29-33, wherein the PPARδ agonist compound is:
(E)-[4-[3-(4-Fluorophenyl)-3-[4-[3-(morpholin-4-yl)propynyl]phenyl]allyloxy]-2-methyl -phenoxy]aceti c acid;
(Z)-[2-Methyl-4-[3-(4-methylphenyl)-3-[4-[3-(morpholin-4-yl)propynyl]phenyl]allyloxy]-phenoxy]acetic acid;
(E)-[2-Methyl-4-[3-[4-[3-(pyrazol -1-yl)prop-1-ynyl]phenyl]-3-(4-trifluoromethylphenyl)-allyloxy]phenoxy]acetic acid;
(E)-[2-Methyl-4-[3-[4-[3-(morpholin-4-yl)propynyl]phenyl]-3-(4-trifluoromethylphenyl)allyloxy]-phenoxy]acetic acid;
(E)-[4-[3-(4-Chlorophenyl)-3-[4-[3-(morpholin-4-yl)propynyl]phenyl]allyl oxy]-2-methyl-phenoxy]aceti c acid;
(E)-[4-[3-(4-Chlorophenyl)-3-[4-[3-(morpholin-4-yl)propynyl]phenyl]allyl oxy]-2-methylphenyl]-propionic acid;
{4-[3-Isobutoxy-5-(3-morpholin-4-yl-prop-1-ynyl)-benzylsulfanyl]-2-methyl-phenoxy}-acetic acid;
{4-[3-Isobutoxy-5-(3-morpholin-4-yl-prop-1-ynyl)-phenylsulfanyl]-2-methyl-phenoxy}-acetic acid; or
{4-[3,3-Bis-(4-bromo-phenyl)-allyloxy]-2-methyl-phenoxy}-acetic acid;
or a pharmaceutically acceptable salt thereof.

39. The method of any one of claims 29-33, wherein the PPARδ agonist is:
(E)-[4-[3-(4-Fluorophenyl)-3-[4-[3-(morpholin-4-yl)propynyl]phenyl]allyloxy]-2-methyl-phenoxy]acetic acid;
(Z)-[2-Methyl-4-[3-(4-methylphenyl)-3-[4-[3-(morpholin-4-yl)propynyl]phenyl]allyloxy]-phenoxy]acetic acid;
(E)-[2-Methyl-4-[3-[4-[3-(pyrazol -1-yl)prop-1-ynyl]phenyl]-3-(4-trifluoromethylphenyl)-allyloxy]phenoxy]acetic acid;
(E)-[2-Methyl-4-[3-[4-[3-(morpholin-4-yl)propynyl]phenyl]-3-(4-trifluoromethylphenyl)allyloxy]-phenoxy]acetic acid;
(E)-[4-[3-(4-Chlorophenyl)-3-[4-[3-(morpholin-4-yl)propynyl]phenyl]allyloxy]-2-methyl-phenoxy]acetic acid;
(E)-[4-[3-(4-Chlorophenyl)-3-[4-[3-(morpholin-4-yl)propynyl]phenyl]allyloxy]-2-methylphenyl]-propionic acid;
{4-[3-Isobutoxy-5-(3-morpholin-4-yl -prop-1-ynyl)-benzylsulfanyl]-2-methyl-phenoxy}-acetic acid;
{4-[3-Isobutoxy-5-(3-morpholin-4-yl -prop-1-ynyl)-phenylsulfanyl]-2-methyl-phenoxy}-acetic acid;
{4-[3,3-Bis-(4-bromo-phenyl)-allyloxy]-2-methyl-phenoxy}-acetic acid;
(R)-3-methyl-6-(2-((5-methyl-2-(4-(trifluoromethyl)phenyl)-1H-imidazol-1-yl)methyl)phenoxy)hexanoic acid;
(R)-3-methyl-6-(2-((5-methyl-2-(6-(trifluoromethyl)pyridin-3-yl)-1H-imidazol-1-yl)methyl)phenoxy)hexanoic acid;
2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954);
2-[2-methyl-4-[[3-methyl-4-[[4-(trifluoromethyl)phenyl]methoxy]phenyl]thio]phenoxy]-acetic acid;
2-[2-methyl-4-[[[4-methyl-2[4-(trifluoromethyl)phenyl]-5-thiazolyl]methyl]thio]phenoxy]-acetic acid (GW-501516);
[4-[[[2-[3-Fluoro-4-(trifluoromethyl)phenyl]-4-methyl-5-thiazolyl]methyl]thio]-2-methylphenoxy]acetic acid (GW0742 also known as GW610742);
2-[2,6 dimethyl-4-[3-[4-(methylthio)phenyl]-3-oxo-1(E)-propenyl]phenoxyl]-2-methylpropanoic acid (elafibranor; GFT-505);
{2-methyl-4-[5-methyl-2-(4-trifluoromethyl-phenyl)-2H-[1,2,3]triazol-4-ylmethylsulfanyl]-phenoxy}-acetic acid;
[4-({(2R)-2-Ethoxy-3-[4-(trifluoromethyl)phenoxy]propyl}sulfanyl)-2-methylphenoxy]acetic acid (seladelpar; MBX-8025);
(S)-4-[cis-2,6-dimethyl-4-(4-trifluoromethoxy-phenyl)piperazine-1-sulfonyl]-indan-2-carboxylic acid or a tosylate salt thereof (KD-3010);
(2s)-2-{4-butoxy-3-[({[2-Fluoro-4-(Trifluoromethyl)phenyl]carbonyl}amino)methyl]benzyl}butanoic acid (TIPP-204);
[4-[3-(4-Acetyl-3-hydroxy-2-propylphenoxy)propoxy]phenoxy]acetic acid (L-165,0411);
2-(4-{2-[(4-Chlorobenzoyl)amino]ethyl}phenoxy)-2-methylpropanoic acid (bezafibrate);
2-(2-methyl-4-(((2-(4-(trifluoromethyl)phenyl)-2H-1,2,3-triazol-4-yl)methyl)thio)phenoxy)acetic acid; or
(R)-2-(4-((2-ethoxy-3-(4-(trifluoromethyl)phenoxy)propyl)thio)phenoxy)acetic acid;
or a pharmaceutically acceptable salt thereof.

40. The method of any one of claims 29-33, wherein:
the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof.

41. A method for treating kidney disease in a mammal, comprising administering to the mammal (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, wherein the kidney disease is polycystic kidney disease (PKD), IgA nephropathy (Bergers Disease), diabetic nephropathy, focal segmental glomerulosclerosis (FSGS), Fabry Disease, Alport syndrome, Glomerulonephritis, Goodpasture syndrome, thin basement membrane nephropathy (TBMN), Nephrotic Syndrome, focal segmental glomerulosclerosis (FSGS), benign familial hematuria (BFH), post-transplant anti-GBM (Glomerular Basement Membrane) nephritis, chronic kidney disease (CKD) or acute kidney injury.

42. A method for treating kidney fibrosis in a mammal, comprising administering to the mammal (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof.

43. The method of any one of claims 40-42, wherein:
(E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, is administered to the mammal at a dose of about 10 mg to about 500 mg.

44. The method of any one of claims 40-42, wherein:
(E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, is administered to the mammal at a dose of about 50 mg to about 200 mg.

45. The method of any one of claims 40-42, wherein:
(E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, is administered to the mammal at a dose of about 75 mg to about 125 mg.

46. The method of any one of claims 40-45, wherein:
the method comprises reducing proteinuria, suppressing the increase of blood urea nitrogen (BUN), reducing intraglomerular pressure, ameliorating glomerular injury, ameliorating extracellular matrix deposition, reducing renal fibrosis, arresting a decline in the estimated glomerular filtration rate (eGFR), increasing eGFR, delaying the onset of end-stage renal disease (ESRD), or combinations thereof.

47. The method of any one of claims 40-45, wherein:
the method comprises achieving a urine protein:creatinine ratio of less than about 0.5 mg/mg if the baseline value is greater than about 1.0 mg/mg.

48. The method of any one of claims 40-45, wherein:
the method comprises achieving an about 50% reduction of urine protein:creatinine ratio if the baseline value is greater than about 0.2 but less than about 1.0.

49. The method of any one of claims 40-48, wherein:
the PPARδ agonist is systemically administered to the mammal.

50. The method of any one of claims 40-49, wherein:
the PPARδ agonist is administered to the mammal orally, by injection or intraveneously.

51. The method of claim 50, wherein:
the PPARδ agonist is administered to the mammal in the form of an oral solution, oral suspension, powder, pill, tablet or capsule.

52. The method of any one of claims 40-51, further comprising:
administering at least one additional therapeutic to the mammal.

53. The method of claim 52, wherein:
the at least one additional therapeutic agent is a Nicotinamide Adenine Dinucleotide (NAD+) pathway modulator.

54. The method of claim 52, wherein:
the at least one additional therapeutic agent is a Poly ADP Ribose Polymerase (PARP) modulator, Aminocarboxymuconate Semialdehyde Decarboxylase (ACMSD) modulator or N′-Nicotinamide Methyltransferase (NNMT) modulator.

55. The method of claim 52, wherein:
the at least one additional therapeutic agent is an inhibitor of the renin-angiotensin-aldosterone system (RAAS).

56. The method of claim 52, wherein:
the at least one additional therapeutic agent is an angiotensin-converting enzyme (ACE) inhibitor, angiotensin-receptor blocker (ARB), aldosterone inhibitor, calcineurin inhibitor, TGF-β1 inhibitor, matrix metalloproteinase inhibitor, vasopeptidase A inhibitor or HMG-CoA reductase inhibitor, chemokine receptor 1 blocker.

57. The method of claim 56, wherein:
the angiotensin converting enzyme (ACE) inhibitor is benazepril, cilazapril, enalapril, fosinopril, lisinopril, perinopril, ramapril, quinapril, or trandolapril;
the angiotensin-receptor blocker (ARB) is candesartan, epresartan, irbesartan, losartan, telmisartan, or valsartan;
the aldosterone inhibitor is spironolactone.

58. The method of any one of claims 40-57, wherein:
the mammal is a human.
